SEQUENCE LISTING (140 sequences)

SEQ ID NO: 1 - DNA, 92 bp, synthetic construct
  source 1..92: mol_type = other DNA; note = Spacer
agctctggagcctgacaaggaggacaggagagatgctgcaagcccaagaagctctctgctcagcctgtcacaacctactgactgccagggca

SEQ ID NO: 2 - RNA, 86 nt, synthetic construct
  source 1..86: mol_type = other RNA; note = WT miR-144
tggggccctggctgggatatcatcatatactgtaagtttgcgatgagacactacagtatagatgatgtactagtccgggcaccccc

SEQ ID NO: 3 - RNA, 66 nt, synthetic construct
  source 1..66: mol_type = other RNA; note = pre-miR-451
cttgggaatggcaaggaaaccgttaccattactgagttcagtaatggtaacggttctcttgctata

SEQ ID NO: 4 - RNA, 86 nt, synthetic construct
  source 1..86: mol_type = other RNA; note = miR-144 mutant
tggggccctggctgggatttcatcatatactgtaagtttgcgatgagacactacagtatagatgatgtactagtccgggcaccccc

SEQ ID NO: 5 - DNA, 1662 bp, synthetic construct
  source 1..1662: mol_type = other DNA; note = CAG promotor
tgtaggaaaaagaagaaggcatgaacatggttagcagaggctctagagccgccggtcacacgccagaagccgaaccccgccctgccccgtcccccccgaaggcagccgtccccccgcggacagccccgaggctggagagggagaaggggacggcggcgcggcgacgcacgaaggccctccccgcccatttccttcctgccggcgccgcaccgcttcgccccgcgcccgctagagggggtgcggcggcgcctcccagatttcggctccgcacagatttgggacaaaggaagtccctgcgccctctcgcacgattaccataaaaggcaatggctgcggcgcgccgcgcctcgacagccgccggcgctccgggggccgccgcgcccctcccccgagccctccccggcccgaggcggccccgccccgcccggcacccccacctgccgccaccccccgcccggcacggcgagccccgcgccacgccccgtacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagccccccccgcaccgcggggcaccgcccccggccgcgctcccctcgcgcacactgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagcccctcaaggctttcacgcagccacagaaaagaaacaagccgtcattaaaccaagcgctaattacagcccggaggagaagggccgtcccgcccgctcacttgtgggagtaacgcggtcagtcagagccggggcgggcggcgcgaggcggcggcggagcggggcacggggcgaaggcagcgtcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactag

SEQ ID NO: 6 - DNA, 798 bp, synthetic construct
  source 1..798: mol_type = other DNA; note = P1 promotor
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 7 - DNA, 877 bp, synthetic construct
  source 1..877: mol_type = other DNA; note = P2 promotor
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 8 - DNA, 133 bp, synthetic construct
  source 1..133: mol_type = other DNA; note = SV40 polyA region
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 9 - DNA, 22 bp, synthetic construct
  source 1..22: mol_type = other DNA; note = miSNCA2 guide strand
tccttggcctttgaaagtcctt

SEQ ID NO: 10 - DNA, 22 bp, synthetic construct
  source 1..22: mol_type = other DNA; note = miSNCA5 guide strand
tgctctttggtcttctcagcca

SEQ ID NO: 11 - DNA, 22 bp, synthetic construct
  source 1..22: mol_type = other DNA; note = miSNCA7 guide strand
tccaacatttgtcacttgctct

SEQ ID NO: 12 - DNA, 22 bp, synthetic construct
  source 1..22: mol_type = other DNA; note = miSNCA12 guide strand
ttggttttctcagcagcagcca

SEQ ID NO: 13 - DNA, 22 bp, synthetic construct
  source 1..22: mol_type = other DNA; note = miSNCA13 guide strand
aacaccctcttttgtctttcct

SEQ ID NO: 14 - DNA, 22 bp, synthetic construct
  source 1..22: mol_type = other DNA; note = miSNCA15 guide strand
ttgcccaactggtcctttttga

SEQ ID NO: 15 - DNA, 22 bp, synthetic construct
  source 1..22: mol_type = other DNA; note = miSNCA16 guide strand
ttaggcttcaggttcgtagtct

SEQ ID NO: 16 - protein, 452 aa, synthetic construct
  source 1..452: note = Amino acid sequence heavy chain of MEDI1341
EVQLLESGGGLVQPGGSLRLSCAASGFTFSSYAMSWVRQAPGKGLEWVSSISHLGGSTYYADSVKGRFTISRDNSKNTLYLQMNSLRAEDTAVYYCAGGANHGKYYYGMDKWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 221 aa, synthetic construct
  source 1..221: note = Amino acid sequence light chain of MEDI1341
QAVLTQPASLSASPGASASLTCTLRSGAPLPKYRIYWYQQKPGSPPQYLLRYKSDADKHQGSGVPSRFSGSKDASANAGILLISGLQSEDEADYYCMVWDHGVWYFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 446 aa, synthetic construct
  source 1..446: note = Amino acid sequence heavy chain of PRX002
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 220 aa, synthetic construct
  source 1..220: note = Amino acid sequence light chain of PRX002
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 22 bp, synthetic construct
  source 1..22: mol_type = other DNA; note = target RNA sequence for miSNCA2
aaggactttcaaaggccaagga

SEQ ID NO: 21 - DNA, 22 bp, synthetic construct
  source 1..22: mol_type = other DNA; note = target RNA sequence for miSNCA5
tggctgagaagaccaaagagca

SEQ ID NO: 22 - DNA, 22 bp, synthetic construct
  source 1..22: mol_type = other DNA; note = target RNA sequence for miSNCA7
agagcaagtgacaaatgttgga

SEQ ID NO: 23 - DNA, 22 bp, synthetic construct
  source 1..22: mol_type = other DNA; note = target RNA sequence for miSNCA12
tggctgctgctgagaaaaccaa

SEQ ID NO: 24 - DNA, 22 bp, synthetic construct
  source 1..22: mol_type = other DNA; note = target RNA sequence for miSNCA13
aggaaagacaaaagagggtgtt

SEQ ID NO: 25 - DNA, 22 bp, synthetic construct
  source 1..22: mol_type = other DNA; note = target RNA sequence for miSNCA15
tcaaaaaggaccagttgggcaa

SEQ ID NO: 26 - DNA, 22 bp, synthetic construct
  source 1..22: mol_type = other DNA; note = target RNA sequence for miSNCA16
agactacgaacctgaagcctaa

SEQ ID NO: 27 - DNA, 92 bp, synthetic construct
  source 1..92: mol_type = other DNA; note = 92 bp spacer region separating mir-451-144 hairpins
acgggaccgtcagtcatccaacactgtccgactcgtctctcgaagaacccgaacgtcgtagagaggacaggaggaacagtccgaggtctcga

SEQ ID NO: 28 - DNA, 423 bp, synthetic construct
  source 1..423: mol_type = other DNA; note = SNCA mRNA 140
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 29 - DNA, 350 bp, synthetic construct
  source 1..350: mol_type = other DNA; note = SNCA mRNA 126
atggatgtattcatgaaaggactttcaaaggccaaggagggagttgtggctgctgctgagaaaaccaaacagggtgtggcagaagcagcaggaaagacaaaagagggtgttctctatgtagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgagga

SEQ ID NO: 30 - DNA, 339 bp, synthetic construct
  source 1..339: mol_type = other DNA; note = SNCA mRNA 112
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaa

SEQ ID NO: 31 - DNA, 297 bp, synthetic construct
  source 1..297: mol_type = other DNA; note = SNCA mRNA 98
atggatgtattcatgaaaggactttcaaaggccaaggagggagttgtggctgctgctgagaaaaccaaacagggtgtggcagaagcagcaggaaagacaaaagagggtgttctctatgtagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaa

SEQ ID NO: 32 - DNA, 72 bp, synthetic construct
  source 1..72: mol_type = other DNA; note = mi451 scaffold
  misc_difference 18..56: note = n may be any nucleotide.
cttgggaatggcaaggtnnnnnnnnnnnnnnnnnnnnnnnnnnnnnnnnnnnnnnntcttgctatacccaga

SEQ ID NO: 33 - DNA, 22 bp, synthetic construct
  source 1..22: mol_type = other DNA; note = miSCR(ANG1) guide strand
gtagttctattagcgcttacta

SEQ ID NO: 34 - DNA, 22 bp, synthetic construct
  source 1..22: mol_type = other DNA; note = miSCR(ANG1) passenger strand
actaaagcgctaatagaactaa

SEQ ID NO: 35 - DNA, 86 bp, synthetic construct
  source 1..86: mol_type = other DNA; note = miR-144 hairpin/scaffold
tggggccctggctgggatttcatcatatactgtaagtttgcgatgagacactacagtatagatgatgtactagtccgggcaccccc

SEQ ID NO: 36 - DNA, 72 bp, synthetic construct
  source 1..72: mol_type = other DNA; note = miSNCA2 mir-451 hairpin/scaffold
cttgggaatggcaaggtccttggcctttgaaagtccttactttcaaaggccaaggctcttgctatacccaga

SEQ ID NO: 37 - DNA, 72 bp, synthetic construct
  source 1..72: mol_type = other DNA; note = miSNCA5 mir-451 hairpin/scaffold
cttgggaatggcaaggtgctctttggtcttctcagccatgagaagaccaaagagcgtcttgctatacccaga

SEQ ID NO: 38 - DNA, 72 bp, synthetic construct
  source 1..72: mol_type = other DNA; note = miSNCA7 mir-451 hairpin/scaffold
cttgggaatggcaaggtccaacatttgtcacttgctctcaagtgacaaatgttggctcttgctatacccaga

SEQ ID NO: 39 - DNA, 72 bp, synthetic construct
  source 1..72: mol_type = other DNA; note = miSNCA12 mir-451 hairpin/scaffold
cttgggaatggcaaggttggttttctcagcagcagccatgctgctgagaaaaccactcttgctatacccaga

SEQ ID NO: 40 - DNA, 72 bp, synthetic construct
  source 1..72: mol_type = other DNA; note = miSNCA13 mir-451 hairpin/scaffold
cttgggaatggcaaggaacaccctcttttgtctttcctaagacaaaagagggtgtctcttgctatacccaga

SEQ ID NO: 41 - DNA, 72 bp, synthetic construct
  source 1..72: mol_type = other DNA; note = miSNCA15 mir-451 hairpin/scaffold
cttgggaatggcaaggttgcccaactggtcctttttgaaaaggaccagttgggcactcttgctatacccaga

SEQ ID NO: 42 - DNA, 72 bp, synthetic construct
  source 1..72: mol_type = other DNA; note = miSNCA16 mir-451 hairpin/scaffold
cttgggaatggcaaggttaggcttcaggttcgtagtcttacgaacctgaagcctactcttgctatacccaga

SEQ ID NO: 43 - DNA, 72 bp, synthetic construct
  source 1..72: mol_type = other DNA; note = miSCR(ANG-1) mir-451 hairpin/scaffold
cttgggaatggcaagggtagttctattagcgcttactaaagcgctaatagaactaatcttgctatacccaga

SEQ ID NO: 44 - DNA, 250 bp, synthetic construct
  source 1..250: mol_type = other DNA; note = mi-cassette
  misc_difference 196..234: note = n may be any nucleotide.
tggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtnnnnnnnnnnnnnnnnnnnnnnnnnnnnnnnnnnnnnnntcttgctatacccaga

SEQ ID NO: 45 - DNA, 250 bp, synthetic construct
  source 1..250: mol_type = other DNA; note = mi-cassette for miSNCA2
tggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccaga

SEQ ID NO: 46 - DNA, 249 bp, synthetic construct
  source 1..249: mol_type = other DNA; note = mi-cassette for miSNCA5
ggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccaga

SEQ ID NO: 47 - DNA, 250 bp, synthetic construct
  source 1..250: mol_type = other DNA; note = mi-cassette for miSNCA7
tggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccaacatttgtcacttgctctcaagtgacaaatgttggctcttgctatacccaga

SEQ ID NO: 48 - DNA, 250 bp, synthetic construct
  source 1..250: mol_type = other DNA; note = mi-cassette for miSNCA12
tggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttggttttctcagcagcagccatgctgctgagaaaaccactcttgctatacccaga

SEQ ID NO: 49 - DNA, 250 bp, synthetic construct
  source 1..250: mol_type = other DNA; note = mi-cassette for miSNCA13
tggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaacaccctcttttgtctttcctaagacaaaagagggtgtctcttgctatacccaga

SEQ ID NO: 50 - DNA, 250 bp, synthetic construct
  source 1..250: mol_type = other DNA; note = mi-cassettefor miSNCA15
tggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccaga

SEQ ID NO: 51 - DNA, 250 bp, synthetic construct
  source 1..250: mol_type = other DNA; note = mi-cassette for miSNCA16
tggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttaggcttcaggttcgtagtcttacgaacctgaagcctactcttgctatacccaga

SEQ ID NO: 52 - DNA, 250 bp, synthetic construct
  source 1..250: mol_type = other DNA; note = mi-cassette cassette for miSCR(ANG-1)
tggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccaga

SEQ ID NO: 53 - DNA, 1386 bp, synthetic construct
  source 1..1386: mol_type = other DNA; note = DNA sequence heavy chain of MEDI1341
gaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgcca

SEQ ID NO: 54 - DNA, 1368 bp, synthetic construct
  source 1..1368: mol_type = other DNA; note = DNA sequence heavy chain of PRX002
gaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgcca

SEQ ID NO: 55 - DNA, 723 bp, synthetic construct
  source 1..723: mol_type = other DNA; note = DNA sequence light chain of MEDI1341
atggcttggacacctctgctgctgctgttcctgagccactgtacaggcagcctgtctcaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 56 - DNA, 729 bp, synthetic construct
  source 1..729: mol_type = other DNA; note = DNA sequence light chain of PRX002
atggacatgagagtgctggctcagctgctgggactgctgctgctttgtttccctggcgctagatgcgacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 57 - protein, 22 aa, synthetic construct
  source 1..22: note = Furin F2A amino acid sequence
VKQTLNFDLLKLAGDVESNPGP

SEQ ID NO: 58 - DNA, 66 bp, synthetic construct
  source 1..66: mol_type = other DNA; note = Furin F2A DNA sequence
gtaaagcagacattaaactttgatttgctgaaacttgcaggtgatgtagagtcaaatccaggtcca

SEQ ID NO: 59 - protein, 449 aa, synthetic construct
  source 1..449: note = Amino acid sequence heavy chain DOB1
EVQLVESGGGLVTFGGSLTLSCAASGFTFSRAWLTWVRQAPGGGLEWVGRILRMADGGATDYAATVKGRFTISRDDSKNTVYLHMNNLKTEDTAVYYCANENFWRLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 217 aa, synthetic construct
  source 1..217: note = Amino acid sequence light chain DOB1
QSALTQPASVSGSPGQSITISCTGTSSDVGTYNHVSWYQQCPGKGPKLLIYDVTNRPSGVSNRFSGSKSDNTASLTISGLQAEDEADYYCYSNTASGAPYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 61 - DNA, 711 bp, synthetic construct
  source 1..711: mol_type = other DNA; note = DNA sequence light chain of DOB1
atggcctgggctctgctgctcctcaccctcctcactcagggcacagggtcctgggcccagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 62 - DNA, 1377 bp, synthetic construct
  source 1..1377: mol_type = other DNA; note = DNA sequence heavy chain of DOB1
gaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgcca

SEQ ID NO: 63 - DNA, 57 bp, synthetic construct
  source 1..57: mol_type = other DNA; note = DNA sequence coding for Ig leader signal peptide
atggaatttggcctgagctggctgttcctggtggccattctgaagggcgtgcagtgt

SEQ ID NO: 64 - protein, 19 aa, synthetic construct
  source 1..19: note = Ig leader signal peptide
MEFGLSWLFLVAILKGVQC

SEQ ID NO: 65 - DNA, 78 bp, synthetic construct
  source 1..78: mol_type = other DNA; note = DNA sequence coding for hGH leader signal peptide
atggcgacgggttcaagaacttccctacttcttgcatttggcctgctttgtttgccgtggttacaggagggctcggca

SEQ ID NO: 66 - protein, 26 aa, synthetic construct
  source 1..26: note = hGH leader signal peptide
MATGSRTSLLLAFGLLCLPWLQEGSA

SEQ ID NO: 67 - DNA, 2253 bp, synthetic construct
  source 1..2253: mol_type = other DNA; note = PRX002 sequence with hGH leader
atggcgacgggttcaagaacttccctacttcttgcatttggcctgctttgtttgccgtggttacaggagggctcggcagaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcagacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 68 - DNA, 2220 bp, synthetic construct
  source 1..2220: mol_type = other DNA; note = PRX002 sequence with Ig leader
atggaatttggcctgagctggctgttcctggtggccattctgaagggcgtgcagtgtgaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggacatgagagtgctggctcagctgctgggactgctgctgctttgtttccctggcgctagatgcgacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 69 - DNA, 2274 bp, synthetic construct
  source 1..2274: mol_type = other DNA; note = MEDI1341 sequence with hGH leader
atggcgacgggttcaagaacttccctacttcttgcatttggcctgctttgtttgccgtggttacaggagggctcggcagaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 70 - DNA, 2232 bp, synthetic construct
  source 1..2232: mol_type = other DNA; note = MEDI1341 sequence with IG leader
atggaatttggcctgagctggctgttcctggtggccattctgaagggcgtgcagtgtgaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcttggacacctctgctgctgctgttcctgagccactgtacaggcagcctgtctcaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 71 - DNA, 2253 bp, synthetic construct
  source 1..2253: mol_type = other DNA; note = DOB1 sequence with hGH leader
atggcgacgggttcaagaacttccctacttcttgcatttggcctgctttgtttgccgtggttacaggagggctcggcagaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 72 - DNA, 2211 bp, synthetic construct
  source 1..2211: mol_type = other DNA; note = DOB1 sequence with Ig leader
atggagtttgggctgagctggattttccttgctgctattttaaaaggtgtccagtgtgaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcctgggctctgctgctcctcaccctcctcactcagggcacagggtcctgggcccagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 73 - DNA, 4911 bp, synthetic construct
  source 1..4911: mol_type = other DNA; note = ScFv-Fc-MEDI1341
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggcgacgggttcaagaacttccctacttcttgcatttggcctgctttgtttgccgtggttacaggagggctcggcagaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctggtggcggaggatctggcggaggtggaagcggcggaggcggatctatggcaacagggagccgaacctctctgctccttgctttcgggctcctttgcctaccgtggctccaagagggctcggcacaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 74 - DNA, 4887 bp, synthetic construct
  source 1..4887: mol_type = other DNA; note = ScFv-Fc-PRX002
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctggtggcggaggatctggcggaggtggaagcggcggaggcggatctatggcgacgggttcaagaacttccctacttcttgcatttggcctgctttgtttgccgtggttacaggagggctcggcagacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 75 - DNA, 4890 bp, synthetic construct
  source 1..4890: mol_type = other DNA; note = ScFv-Fc-DOB1
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcaggtggcggaggatctggcggaggtggaagcggcggaggcggatctatggcaacagggagccgaacctctctgctccttgctttcgggctcctttgcctaccgtggctccaagagggctcggcacagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 76 - DNA, 562 bp, synthetic construct
  source 1..562: mol_type = other DNA; note = Alt promoter sequence
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgg

SEQ ID NO: 77 - DNA, 384 bp, synthetic construct
  source 1..384: mol_type = other DNA; note = intron sequence ("NNN" refers to where the miQURE cassette can be inserted)
  misc_difference 185..193: note = n may be any nucleotide.
gcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatnnnnnnnnnaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgg

SEQ ID NO: 78 - DNA, 3919 bp, synthetic construct
  source 1..3919: mol_type = other DNA; note = miCTR1
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggggcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 79 - DNA, 5920 bp, synthetic construct
  source 1..5920: mol_type = other DNA; note = miSNCA5+MEDI1341-IgLeader
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggaatttggcctgagctggctgttcctggtggccattctgaagggcgtgcagtgtgaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcttggacacctctgctgctgctgttcctgagccactgtacaggcagcctgtctcaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 80 - DNA, 5908 bp, synthetic construct
  source 1..5908: mol_type = other DNA; note = miSNCA5+PRX-IgLeader
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggaatttggcctgagctggctgttcctggtggccattctgaagggcgtgcagtgtgaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggacatgagagtgctggctcagctgctgggactgctgctgctttgtttccctggcgctagatgcgacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 81 - DNA, 5962 bp, synthetic construct
  source 1..5962: mol_type = other DNA; note = miSNCA5+MEDI1341-hGH-SP
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggcgacgggttcaagaacttccctacttcttgcatttggcctgctttgtttgccgtggttacaggagggctcggcagaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 82 - DNA, 5941 bp, synthetic construct
  source 1..5941: mol_type = other DNA; note = miSNCA5_ PRX002-hGH-SP
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcagacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 83 - DNA, 5899 bp, synthetic construct
  source 1..5899: mol_type = other DNA; note = miCTR1+DOB1-IGleader
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggagtttgggctgagctggattttccttgctgctattttaaaaggtgtccagtgtgaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcctgggctctgctgctcctcaccctcctcactcagggcacagggtcctgggcccagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 84 - DNA, 5941 bp, synthetic construct
  source 1..5941: mol_type = other DNA; note = miCTR1+DOB1-hGH-SP
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 85 - DNA, 12389 bp, synthetic construct
  source 1..12389: mol_type = other DNA; note = pVD1562 (vec-Ab-MEDI1341-IgL)
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggaatttggcctgagctggctgttcctggtggccattctgaagggcgtgcagtgtgaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcttggacacctctgctgctgctgttcctgagccactgtacaggcagcctgtctcaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 86 - DNA, 12377 bp, synthetic construct
  source 1..12377: mol_type = other DNA; note = pVD1563 (vec-Ab-PRX002-IgL)
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggaatttggcctgagctggctgttcctggtggccattctgaagggcgtgcagtgtgaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggacatgagagtgctggctcagctgctgggactgctgctgctttgtttccctggcgctagatgcgacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 87 - DNA, 12431 bp, synthetic construct
  source 1..12431: mol_type = other DNA; note = pVD1564 (vec-Ab-MEDI1341-hGH)
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggcgacgggttcaagaacttccctacttcttgcatttggcctgctttgtttgccgtggttacaggagggctcggcagaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 88 - DNA, 12410 bp, synthetic construct
  source 1..12410: mol_type = other DNA; note = pVD1565 (vec-Ab-PRX002-hGH)
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcagacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 89 - DNA, 12368 bp, synthetic construct
  source 1..12368: mol_type = other DNA; note = pVD1566 (vec-Ab-DOB1-IgL)
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggagtttgggctgagctggattttccttgctgctattttaaaaggtgtccagtgtgaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcctgggctctgctgctcctcaccctcctcactcagggcacagggtcctgggcccagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 90 - DNA, 12410 bp, synthetic construct
  source 1..12410: mol_type = other DNA; note = pVD1567 (vec-Ab-DOB1-hGH)
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 91 - DNA, 18 bp, synthetic construct
  source 1..18: mol_type = other DNA; note = primer
gggaggtgtgggaggttt

SEQ ID NO: 92 - DNA, 20 bp, synthetic construct
  source 1..20: mol_type = other DNA; note = primer
aatgattaacccgccatgct

SEQ ID NO: 93 - DNA, 25 bp, synthetic construct
  source 1..25: mol_type = other DNA; note = probe
acttatctacagatctgcggccgct

SEQ ID NO: 94 - DNA, 22 bp, synthetic construct
  source 1..22: mol_type = other DNA; note = primer
agtaacgccaatagggactttc

SEQ ID NO: 95 - DNA, 22 bp, synthetic construct
  source 1..22: mol_type = other DNA; note = primer
ggcgtacttggcatatgataca

SEQ ID NO: 96 - DNA, 24 bp, synthetic construct
  source 1..24: mol_type = other DNA; note = probe
ttacggtaaactgcccacttggca

SEQ ID NO: 97 - DNA, 20 bp, synthetic construct
  source 1..20: mol_type = other DNA; note = primer
aaatgttggaggagcagtgg

SEQ ID NO: 98 - DNA, 20 bp, synthetic construct
  source 1..20: mol_type = other DNA; note = primer
tccagaattccttcctgtgg

SEQ ID NO: 99 - DNA, 21 bp, synthetic construct
  source 1..21: mol_type = other DNA; note = probe
agcagggagcattgcagcagc

SEQ ID NO: 100 - DNA, 22 bp, synthetic construct
  source 1..22: mol_type = other DNA; note = primer
ccggagaacaactacaagacca

SEQ ID NO: 101 - DNA, 21 bp, synthetic construct
  source 1..21: mol_type = other DNA; note = primer
cacggagcatgagaagacgtt

SEQ ID NO: 102 - DNA, 27 bp, synthetic construct
  source 1..27: mol_type = other DNA; note = probe
actcaccgtggacaagagcaggtggca

SEQ ID NO: 103 - DNA, 20 bp, synthetic construct
  source 1..20: mol_type = other DNA; note = primer
cctctgcctaagtaccggat

SEQ ID NO: 104 - DNA, 20 bp, synthetic construct
  source 1..20: mol_type = other DNA; note = primer
accatgcagtagtagtcggc

SEQ ID NO: 105 - DNA, 25 bp, synthetic construct
  source 1..25: mol_type = other DNA; note = probe
acaagagcgacgccgacaagcacca

SEQ ID NO: 106 - DNA, 20 bp, synthetic construct
  source 1..20: mol_type = other DNA; note = primer
gatgacacagagccctagca

SEQ ID NO: 107 - DNA, 20 bp, synthetic construct
  source 1..20: mol_type = other DNA; note = primer
atggtcagggtgaagtcggt

SEQ ID NO: 108 - DNA, 27 bp, synthetic construct
  source 1..27: mol_type = other DNA; note = probe
agcagattttctggcagcggctctggc

SEQ ID NO: 109 - DNA, 4811 bp, synthetic construct
  source 1..4811: mol_type = other DNA; note = miSNCA_5_w144
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgagcggaaggcccatgaggccagttaattaacgagctcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaagtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgcgccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggggcggccgcaagctttagagctcatggcgcgcctaggccttgacggccttccgccaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 110 - DNA, 3584 bp, synthetic construct
  source 1..3584: mol_type = other DNA; note = mi-ab-PRX002-hGH DNA sequence
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcagacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 111 - DNA, 3551 bp, synthetic construct
  source 1..3551: mol_type = other DNA; note = mi-ab-PRX002-IgLeader DNA sequence
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggaatttggcctgagctggctgttcctggtggccattctgaagggcgtgcagtgtgaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggacatgagagtgctggctcagctgctgggactgctgctgctttgtttccctggcgctagatgcgacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 112 - DNA, 3605 bp, synthetic construct
  source 1..3605: mol_type = other DNA; note = mi-ab-MEDI1341-hGH DNA sequence
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggcgacgggttcaagaacttccctacttcttgcatttggcctgctttgtttgccgtggttacaggagggctcggcagaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 113 - DNA, 3563 bp, synthetic construct
  source 1..3563: mol_type = other DNA; note = mi-ab-MEDI1341-IgLeader DNA sequence
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggaatttggcctgagctggctgttcctggtggccattctgaagggcgtgcagtgtgaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcttggacacctctgctgctgctgttcctgagccactgtacaggcagcctgtctcaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 114 - DNA, 3584 bp, synthetic construct
  source 1..3584: mol_type = other DNA; note = mi-ab-DOB1-hGH DNA sequence
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 115 - DNA, 3542 bp, synthetic construct
  source 1..3542: mol_type = other DNA; note = mi-ab-DOB1-IgLeader DNA sequence
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagataatgtttaattacctggagcacctgcctgaaatcactttttttcaggttgggctagcgccaccatggagtttgggctgagctggattttccttgctgctattttaaaaggtgtccagtgtgaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcctgggctctgctgctcctcaccctcctcactcagggcacagggtcctgggcccagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 116 - DNA, 30 bp, synthetic construct
  source 1..30: mol_type = other DNA; note = bp spacer sequence between mir-451-mir-451 hairpins
aaacgtgccaggaagctgactgccagggca

SEQ ID NO: 117 - DNA, 72 bp, synthetic construct
  source 1..72: mol_type = other DNA; note = miR-451-derived scaffold
  misc_difference 17..55: note = n may be any nucleotide.
cttgggaatggcaaggnnnnnnnnnnnnnnnnnnnnnnnnnnnnnnnnnnnnnnnctcttgctatacccaga

SEQ ID NO: 118 - DNA, 86 bp, synthetic construct
  source 1..86: mol_type = other DNA; note = miR-144-derived scaffold
  misc_difference 15..36: note = n may be any nucleotide.
  misc_difference 52..71: note = n may be any nucleotide.
tggggccctggctgnnnnnnnnnnnnnnnnnnnnnntttgcgatgagacacnnnnnnnnnnnnnnnnnnnnagtccgggcaccccc

SEQ ID NO: 119 - DNA, 86 bp, synthetic construct; identical to SEQ ID NO: 35
  source 1..86: mol_type = other DNA; note = mir-144 A >T mutant helper

SEQ ID NO: 120 - DNA, 86 bp, synthetic construct
  source 1..86: mol_type = other DNA; note = human pri-mir-144 sequence
tggggccctggctgggatatcatcatatactgtaagtttgcgatgagacactacagtatagatgatgtactagtccgggcaccccc

SEQ ID NO: 121 - DNA, 14 bp, synthetic construct
  source 1..14: mol_type = other DNA; note = miR-144 scaffold
  misc_difference 14: note = n may be any nucleotide.
tggggccctggctn

SEQ ID NO: 122 - DNA, 15 bp, synthetic construct
  source 1..15: mol_type = other DNA; note = 15 nucleotide sequence
  misc_difference 13: note = n may be any nucleotide.
tttgcgatgaganmm

SEQ ID NO: 123 - DNA, 15 bp, synthetic construct
  source 1..15: mol_type = other DNA; note = follow sequence
agtccgggcaccccc

SEQ ID NO: 124 - DNA, 28 bp, synthetic construct
  source 1..28: mol_type = other DNA; note = follow sequence
atcggcgctatgcttcctgtgcccccag

SEQ ID NO: 125 - DNA, 5625 bp, synthetic construct
  source 1..5625: mol_type = other DNA; note = MEDI1341-IgLeader
gctaataacaggcctgctggtaatcgcaggcctttttatttggtgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgtaggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggaatttggcctgagctggctgttcctggtggccattctgaagggcgtgcagtgtgaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcttggacacctctgctgctgctgttcctgagccactgtacaggcagcctgtctcaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgcctatcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaaataaaacgaaaggcccagtcttgcgactgagcctttcgttttattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaa

SEQ ID NO: 126 - DNA, 5613 bp, synthetic construct
  source 1..5613: mol_type = other DNA; note = PRX002-IgLeader
gctaataacaggcctgctggtaatcgcaggcctttttatttggtgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgtaggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggaatttggcctgagctggctgttcctggtggccattctgaagggcgtgcagtgtgaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggacatgagagtgctggctcagctgctgggactgctgctgctttgtttccctggcgctagatgcgacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgcctatcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaaataaaacgaaaggcccagtcttgcgactgagcctttcgttttattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaa

SEQ ID NO: 127 - DNA, 5604 bp, synthetic construct
  source 1..5604: mol_type = other DNA; note = DOB1-IgLeader
gctaataacaggcctgctggtaatcgcaggcctttttatttggtgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgtaggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggagtttgggctgagctggattttccttgctgctattttaaaaggtgtccagtgtgaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcctgggctctgctgctcctcaccctcctcactcagggcacagggtcctgggcccagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgcctatcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaaataaaacgaaaggcccagtcttgcgactgagcctttcgttttattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaa

SEQ ID NO: 128 - DNA, 5667 bp, synthetic construct
  source 1..5667: mol_type = other DNA; note = MEDI1341-hGH-SP
gctaataacaggcctgctggtaatcgcaggcctttttatttggtgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgtaggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggcgacgggttcaagaacttccctacttcttgcatttggcctgctttgtttgccgtggttacaggagggctcggcagaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctgggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgcctatcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaaataaaacgaaaggcccagtcttgcgactgagcctttcgttttattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaa

SEQ ID NO: 129 - DNA, 5646 bp, synthetic construct
  source 1..5646: mol_type = other DNA; note = PRX002-hGH-SP
gctaataacaggcctgctggtaatcgcaggcctttttatttggtgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgtaggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctgccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcagacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgcctatcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaaataaaacgaaaggcccagtcttgcgactgagcctttcgttttattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaa

SEQ ID NO: 130 - DNA, 5646 bp, synthetic construct
  source 1..5646: mol_type = other DNA; note = DOB1-hGH-SP
gctaataacaggcctgctggtaatcgcaggcctttttatttggtgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgtaggcggaaggccgtcaaggccgcatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcgccaccatggcgacgggttcaagaacttccctacttcttgcatttggcctgctttgtttgccgtggttacaggagggctcggcagaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcagccagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcacagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctaggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatgctttatttgtgaaatttgtgatgctattgctttatttgtaaccattataagctgcaataaacaagttaacaacaacaattgcattcattttatgtttcaggttcagggggaggtgtgggaggttttttaaagcggccgcctgggcctcatgggccttccgcctatcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaaataaaacgaaaggcccagtcttgcgactgagcctttcgttttattgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaa

SEQ ID NO: 131 - DNA, 22 bp, synthetic construct
  source 1..22: mol_type = other DNA; note = miSNCA2 passenger strand
ccttactttcaaaggccaaggc

SEQ ID NO: 132 - DNA, 22 bp, synthetic construct
  source 1..22: mol_type = other DNA; note = miSNCA5 passenger strand
gccatgagaagaccaaagagcg

SEQ ID NO: 133 - DNA, 22 bp, synthetic construct
  source 1..22: mol_type = other DNA; note = miSNCA7 passenger strand
ctctcaagtgacaaatgttggc

SEQ ID NO: 134 - DNA, 22 bp, synthetic construct
  source 1..22: mol_type = other DNA; note = miSNCA12 passenger strand
gccatgctgctgagaaaaccac

SEQ ID NO: 135 - DNA, 22 bp, synthetic construct
  source 1..22: mol_type = other DNA; note = miSNCA13 passenger strand
tcctaagacaaaagagggtgtc

SEQ ID NO: 136 - DNA, 22 bp, synthetic construct
  source 1..22: mol_type = other DNA; note = miSNCA15 passenger strand
ttgaaaaggaccagttgggcac

SEQ ID NO: 137 - DNA, 22 bp, synthetic construct
  source 1..22: mol_type = other DNA; note = miSNCA16 passenger strand
gtcttacgaacctgaagcctac

SEQ ID NO: 138 - DNA, 1611 bp, synthetic construct
  source 1..1611: mol_type = other DNA; note = ScFv-Fc version of MEDI1341
atggcgacgggttcaagaacttccctacttcttgcatttggcctgctttgtttgccgtggttacaggagggctcggcagaagtgcagctgctggaatctggcggaggactggttcaacctggcggctctctgagactgtcttgtgccgccagcggcttcacctttagcagctacgccatgagctgggtccgacaggctcctggcaaaggccttgaatgggtgtccagcatctctcacctcggcggcagcacctactacgccgattctgtgaagggcagattcaccatcagccgggacaacagcaagaacaccctgtacctgcagatgaacagcctgagagccgaggacaccgccgtgtactattgtgccggcggagccaatcacggcaagtactactacggcatggataagtggggccagggcaccaccgtgacagtctcttctggtggcggaggatctggcggaggtggaagcggcggaggcggatctatggcaacagggagccgaacctctctgctccttgctttcgggctcctttgcctaccgtggctccaagagggctcggcacaggctgttctgactcagcctgccagcctgtctgcttctcctggcgcttctgcctctctgacctgcacacttagatctggcgcccctctgcctaagtaccggatctactggtatcagcagaagcctggcagccctcctcagtacctgctgagatacaagagcgacgccgacaagcaccaaggcagcggagtgcctagcagattcagcggctctaaggacgcctctgccaacgccggaatcctgctgatttctggcctgcagtctgaggacgaggccgactactactgcatggtctgggatcacggcgtgtggtactttggcggcggaacaaagctgaccgtgctggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaatga

SEQ ID NO: 139 - DNA, 1587 bp, synthetic construct
  source 1..1587: mol_type = other DNA; note = ScFv-Fc version of PRX002
atggcgacgggttcaagaacttccctacttcttgcatttggcctgctttgtttgccgtggttacaggagggctcggcagaggtgcagctggttgaatctggcggaggactggttcagcctggcggatctctgagactgtcttgtgccgccagcggcttcaccttcagcaactacggcatgagctgggtccgacaggcccctggaaaaggccttgaatgggtcgccagcatctctagcggcggaggcagcacctactatcccgacaatgtgaagggcagattcaccatcagccgggacgacgccaagaacagcctgtacctgcagatgaactccctgagagccgaggacaccgccgtgtactattgtgctagaggcggagccggcatcgactattggggacagggaacactggtcaccgtgtcctctggtggcggaggatctggcggaggtggaagcggcggaggcggatctatggcgacgggttcaagaacttccctacttcttgcatttggcctgctttgtttgccgtggttacaggagggctcggcagacatccagatgacacagagccctagcagcctgtctgccagcgtgggagacagagtgaccatcacatgcaagagcatccagacactgctgtactccagcaaccagaagaactacctggcctggttccagcagaagcctggcaaggctcctaagctgctgatctactgggccagcatcagaaagtccggcgtgcccagcagattttctggcagcggctctggcaccgacttcaccctgaccatatctagcctgcagcctgaggacctggccacctactactgccagcagtactacagctaccctctgacctttggcggaggcaccaagctggaaatcaaggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaatga

SEQ ID NO: 140 - DNA, 1590 bp, synthetic construct
  source 1..1590: mol_type = other DNA; note = ScFv-Fc version of DOB1
atggcgacgggttcaagaacttccctacttcttgcatttggcctgctttgtttgccgtggttacaggagggctcggcagaggtgcagctggtggagtctgggggaggcttggtgacttttggggggtcccttacactctcctgtgcagcctctggattcaccttcagtcgtgcctggctgacgtgggtccgccaggctccagggggggggctggagtgggttggccgtattctaaggatggctgatggtggggcaacagactacgctgcaaccgttaaaggcagattcaccatctcaagagatgattcaaaaaacacagtttatctgcacatgaacaacctgaaaaccgaagacacagccgtgtattactgtgccaacgaaaacttttggcgccttgacaactggggccagggaaccctggtcaccgtctcctcaggtggcggaggatctggcggaggtggaagcggcggaggcggatctatggcaacagggagccgaacctctctgctccttgctttcgggctcctttgcctaccgtggctccaagagggctcggcacagtctgcgctgactcagccggcctctgtgtctgggtctcctggacagtcgatcaccatctcctgcactggaaccagcagtgatgttgggacttataaccatgtctcctggtaccaacagtgcccaggcaaaggccccaagctcttgatttatgatgtcactaatcggccctcaggggtttctaatcgcttctctggctccaagtctgacaacacggcctcgctgaccatctctgggctccaggctgaggacgaggctgattattactgctactcaaatacagccagcggcgctccttatgtcttcggcagtgggaccaaggtcaccgtcctagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaatcaagtcagcctgacctgcctggtcaaaggcttctatcccagcgacatcgccgtggagtgggagagcaatgggcagccggagaacaactacaagaccacgcctcccgtgctggactccgacggctccttcttcctctactcaaaactcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2026/0286387 A1
(43) Pub. Date: Sep. 24, 2026

(54) Gene delivery vehicles comprising RNA and antibodies

(71) Applicant: UNIQURE BIOPHARMA B.V., Amsterdam (NL)

(72) Inventors: Astrid Valles-Sanchez, Amsterdam (NL); Seyda Acar Broekmans, Amsterdam (NL); Sebastian Kieper, Amsterdam (NL); - Anggakusuma, Amsterdam (NL)

(73) Assignee: UNIQURE BIOPHARMA B.V., Amsterdam (NL)

(21) Appl. No.: 19/473,495

(22) PCT Filed: Apr. 18, 2024
(86) PCT No.: PCT/EP2024/060521

§ 371 Date: Oct. 8, 2025

(30) Foreign Application Priority Data
Apr. 18, 2023 (EP) 23168449.9

(51) Int. Cl. C12N 15/86; C07K 16/18; C12N 15/11

(52) U.S. Cl. CPC C12N 15/86; C07K 16/18; C12N 15/11; C07K 2317/24; C07K 2317/622; C12N 2310/20; C12N 2310/531; C12N 2750/14122; C12N 2750/14143; C12N 2830/50; C12N 2830/60

(57) ABSTRACT

The present invention relates to a gene delivery vehicle comprising at least one nucleic acid comprising a sequence encoding a first RNA and a sequence encoding a second RNA, wherein the second RNA comprises a guide sequence and wherein the first and second and optionally third RNA each comprise a hairpin and encoding a full-length mAb or an antigen binding fragment thereof.

### FIELD OF INVENTION

The present invention relates to the fields of biotechnology, medicine and gene therapy. The invention relates to a gene delivery vehicle comprising at least one nucleic acid comprising a sequence encoding a first RNA and a sequence encoding a second RNA, wherein the second RNA comprises a guide sequence and wherein the first and second RNA each comprise a hairpin and encoding a full-length mAb or an antigen binding fragment thereof, pharmaceutical composition and uses in treatment thereof.

### BACKGROUND OF THE INVENTION

Central nervous system (CNS) or neurological disorders are very serious problems and major health concerns all over the world. The most challenging of the CNS diseases are neurodegenerative diseases, characterized by age-related gradual decline in neurological function, often accompanied by neuronal death. Synucleinopathies, Huntington's and tauopathies are examples of neurodegenerative diseases and have been well described in terms of disease mechanisms and pathology.

As an example, synucleinopathies are characterized by aggregation of alpha-Synuclein protein (α-Syn), encoded by the SNCA (Synuclein Alpha) gene. Aggregated α-Syn is one of the main components of Lewy Bodies (LBs), the key neuropathological hallmarks in Parkinson's disease (PD) and Lewy Body Dementia (LBD) and of Glial Cytoplasmic Inclusions (GCI) a hallmark in multiple system atrophy (MSA). The α-Syn aggregates may spread in a prion-like manner, with toxic aggregated forms being transmitted from cell-to-cell as the disease progresses.

While some symptomatic therapies are available, successful treatment strategies for neurodegenerative diseases have so far been limited. With an increase in lifespan and changing population demographics, the incidence of CNS diseases is expected to increase significantly in the 21st century.

Thus there remains a great need for new strategies that allow the treatment of CNS diseases.

### SUMMARY OF INVENTION

In a first aspect the invention provides for a gene delivery vehicle comprising an expression cassette comprising at least one nucleic acid comprising a sequence encoding a first RNA and a sequence encoding a second RNA, wherein the second RNA comprises a guide sequence and wherein the first and second RNA each comprise a hairpin, and encoding a full-length mAb or an antigen binding fragment thereof, wherein the guide sequence is substantially complementary to a target sequence in a gene associated with a disease and wherein the mAb or antigen binding fragment thereof is targeting a protein associated with that same disease.

In a second aspect the invention provides for a combination comprising:
a first gene delivery vehicle comprising a first expression cassette comprising at least one nucleic acid comprising a sequence encoding a first RNA and a sequence encoding a second RNA,
wherein the second RNA comprises a guide sequence and wherein the first and second RNA each comprise a hairpin; and
a second gene delivery vehicle comprising a second expression cassette, comprising a transgene encoding a full-length mAb or an antigen binding fragment thereof,
   wherein the guide sequence is substantially complementary to a target sequence in a gene associated with a disease and wherein the mAb or antigen binding fragment thereof is targeting a protein associated with that same disease.

In a further aspect, the invention provides for a pharmaceutical composition comprising the gene delivery vehicle of the invention or the combination according to the invention and at least one pharmaceutically acceptable excipient.

In yet a further aspect, the invention provides for gene delivery vehicle of the invention or the combination according to the invention for use as a medicament, preferably for use of a medicament in the treatment of a CNS disease.

### DETAILED DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

For the purposes of the present invention, the term “obtained” is considered to be a preferred embodiment of the term “obtainable”. If hereinafter e.g. an antibody is defined to be obtainable from a specific source, this is also to be understood to disclose an antibody that is obtained from this source.

As used herein, the term “and/or” indicates that one or more of the stated cases may occur, alone or in combination with at least one of the stated cases, up to with all of the stated cases. As used herein the term “at least one of” has the same meaning, i.e., that one or more of the stated cases may occur, alone or in combination with at least one of the stated cases, up to with all of the stated cases.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value. As used herein, “an effective amount” is meant the amount of an agent required to ameliorate the symptoms of a disease relative to an untreated patient. The effective amount of active agent(s) used to practice the present invention for therapeutic treatment of, for example a cancer or a neurologic disease, varies depending upon the manner of administration, the age, body weight, and general health of the subject. Ultimately, the attending physician or veterinarian will decide the appropriate amount and dosage regimen. Such amount is referred to as an “effective” amount, which may be determined for instance as genome copies per kilogram (GC/kg) or as GC per dose. Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptides or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimum percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins, the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

Within the context of the present invention, the term “variant thereof”, when referring to a given sequence (SEQ ID), includes any nucleic acids which retain at least some of the properties of the corresponding native nucleic, for example, reduction of RNA expression. The term “variant” may include any nucleic acids with at least 50; 55; 60; 65; 70; 75; 80; 90; or 95% sequence identity with the native nucleic acid.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C. Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly-A sequence (such as the 3′ terminal poly(A) tract of mRNAs), or to a complementary stretch of T (or U) residues, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting the expression of a gene in host cells or host organisms compatible with such sequences. These expression vectors typically include at least one “expression cassette” that is the functional unit capable of affecting the expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in effecting expression may also be present, such as expression enhancer elements. The expression vector will be introduced into a suitable host cell and be able to affect the expression of the coding sequence in an in vitro cell culture of the host cell. A preferred expression vector will be suitable for the expression of viral proteins and/or nucleic acids, particularly recombinant AAV proteins and/or nucleic acids.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter the expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences that affect the transcription and translation stability, e.g., promoters, as well as sequences that affect the translation, e.g., Kozak sequences, are known in insect cells. Expression control sequences can be of such nature as to modulate the nucleotide sequence to which it is operably linked such that lower expression levels or higher expression levels are achieved.

As used herein the term “codon optimization” refers to experimental approaches designed to improve the codon composition of a recombinant gene based on various criteria without altering the amino acid sequence. This is possible because most amino acids are encoded by more than one codon. Most codon-optimization approaches avoid the use of rare codons. However, different approaches vary in the extent of other features considered, including mRNA elements that can inhibit expression, nucleotide context of the initiation codon, mRNA secondary structures, sequence repeats, nucleotide composition, internal ribosome entry sites, promoter sequences, and putative splice donor and acceptor sites. In addition, some programs consider protein structural information, intragenic poly(A) sites, stop codons in alternative reading frames, and dinucleotides that are targets for RNase cleavage, mutation, and methylation-dependent gene silencing. The person skilled in the art has within their understanding the requirements needed to design such a codon-optimised nucleic acid.

In synucleinopathies, α-Syn aggregates may spread in a prion-like manner, with toxic aggregated forms being transmitted from cell-to-cell as the disease progresses. This toxic cycle can be halted by decreasing the α-Syn protein levels or by facilitating the clearance of the α-Syn aggregates outside of the cell. This toxic cycle can be halted by decreasing the α-Syn protein levels or by facilitating the clearance of the α-Syn aggregates outside of the cell. However to improve treatment efficacy, the inventors of present application hypothesized that it would be more efficient to target both the α-Syn protein levels while simultaneously clearing the α-Syn aggregates outside of the cell. Many neurodegenerative diseases have a similar pathology in that aberrant proteins form toxic aggregates in neurons, such as Alzheimer, Huntington's disease, Amyotrophic Lateral Sclerosis, and others. To this end, the inventors developed a gene delivery vehicle that allows to target both the expression of an aberrant protein as well as the already expressed proteins at the same time.

Accordingly, in a first aspect the invention provides for a gene delivery vehicle comprising an expression cassette:
comprising at least one nucleic acid comprising a sequence encoding a first RNA and a sequence encoding a second RNA, wherein the second RNA comprises a guide sequence and wherein the first and second RNA each comprise a hairpin, and
encoding a full-length mAb or an antigen binding fragment thereof,
wherein the guide sequence is substantially complementary to a target sequence in a gene associated with a disease and wherein the mAb or antigen binding fragment thereof is targeting a protein associated with that same disease.

As explained herein, the gene delivery vehicle of the invention comprises an expression cassette comprising at least one nucleic acid encoding an RNA that comprises a guide sequence that is substantially complementary to a target sequence in a gene associated with a disease and encodes an antibody of antigen binding fragment thereof that is targeting a protein associated with that same disease. An exemplary list of target sequences and proteins causing the same disease is provided in Table 1. The gene delivery system as described herein is thus capable of targeting a disease intracellularly (by lowering mRNA levels in the cell through the use of miRNAs) and extracellularly (by targeting the proteins secreted by the cell) at the same time.

A gene delivery vehicle as used herein encompasses but is not limited to a vector, and can be a plasmid, cosmid, phagemid, bacterial artificial chromosome (BAC), or a viral vector. In certain embodiments the gene delivery vehicle is a viral vector such as lentiviral vectors, retrotransposon based vector systems, or AAV vectors. It is understood that as e.g. lentiviral vectors carry an RNA genome, the RNA genome will encode for the said expression cassette such that after transduction of a cell, the said DNA sequence and said expression cassette is formed.

The gene delivery vehicle according to the invention comprises an expression cassette comprising at least one nucleic acid comprising a sequence encoding a first RNA and a sequence encoding a second RNA, wherein the second RNA comprises a guide sequence and wherein the first and second RNA each comprise a hairpin. The term “nucleic acid” as used herein takes its regular meaning in the art. Thus, the term “RNA” or “RNA molecule” or “ribonucleic acid molecule” as used herein refers to a polymer of ribonucleotides (e.g., 2, 3, 4, 5, 10, 15, 20, 25, 30, or more ribonucleotides) and the term “DNA” or “DNA molecule” or “deoxyribonucleic acid molecule” as used herein refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA. In some embodiments, the sequence of a nucleic acid of the invention is codon optimised.

As used herein, the term “small interfering RNA” (“siRNA”) (also referred to in the art as “short interfering RNAs”) refers to an RNA (or RNA analogue) comprising between about 10-50 nucleotides (or nucleotide analogues) which is capable of directing or mediating RNA interference. Preferably, an siRNA comprises between about 15-30 nucleotides or nucleotide analogues, more preferably between about 16-25 nucleotides (or nucleotide analogues), even more preferably between about 18-23 nucleotides (or nucleotide analogues), and even more preferably between about 19-22 nucleotides (or nucleotide analogues) (e.g., 19, 20, 21 or 22 nucleotides or nucleotide analogues). The term “short” siRNA refers to a siRNA comprising about 21 nucleotides (or nucleotide analogues), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be induced, for example, to silence the expression of target genes. Double stranded RNA structures that are suitable for inducing RNAi are well known in the art. For example, a small interfering RNA (siRNA) can induce RNAi. An siRNA comprises two separate RNA strands, one strand comprising a first RNA sequence and the other strand comprising a second RNA sequence, thus a first and a second strand. An siRNA design that is often used involves consecutive base pairs with a 3′ overhang. The first and/or second strand may comprise a 3-overhang. The 3-overhang preferably is a dinucleotide overhang on both strands of the siRNA. Such a design is based on observed Dicer processing of larger double stranded RNAs that results in siRNAs having these features. The 3-overhang may be comprised in the first strand. The 3-overhang may be in addition to the first strand. The length of the two strands of which an siRNA is composed may be 19, 20, 21, 25 22, 23, 24, 25, 26 or 27 nucleotides or more.

siRNAs may also serve as Dicer substrates. For example, a Dicer substrate may be a 27-mer consisting of two strands of RNA that have 27 consecutive base pairs. The first strand is positioned at the 3′-end of the 27-mer duplex. At the 3′-ends, like with siRNAs, each or one of the strands may comprise a two nucleotide overhang. The 3-overhang may be comprised in the first strand. The 3-overhang may be in addition to the first strand. 5′ from the first strand, additional sequences may be included that are either complementary to the target RNA sequence adjacent or not. The other end of the siRNA dicer substrate is blunt ended. This dicer substrate design may result in a preference in processing by Dicer such that an siRNA can be formed like the siRNA design as described above, having 19 consecutive base pairs and 2 nucleotide overhangs at both 3′-ends. In any case, siRNAs, or the like, are composed of two separate RNA strands (Fire et al. 1998, Nature. 1998 Feb. 19; 391 (6669):806-1 1) each RNA strand comprising or consisting of the first and second RNA strand in accordance with to the invention. Thus, the nucleic acid of the invention may be said to derive a first and second RNA strand, which is the first RNA, and a third and fourth RNA strand, which is the second RNA. Alternative naming conventions for each first, second, third and fourth RNA strand are within the scope of the invention, which may be complementary, substantially complementary, or unique to each other, or in any other required arrangement as discussed herein.

The loop sequence may also be a stem-loop sequence, whereby the double stranded region of the shRNA is extended. Like the siRNA dicer substrate as described above, an shRNA can be processed by e.g. Dicer to provide for an siRNA having an siRNA design such as described above, having e.g. 19 consecutive base pairs and 2 nucleotide overhangs at both 3′-ends. In case the shRNA is to be processed by Dicer, it is preferred to have the first and second strands at the end of the shRNA, i.e. such that the putative strands of the siRNA are linked via a stem loop sequence: 5′-first strand-apical loop sequence-second strand-optional 2 nt overhang sequence-3′. Or, conversely, 5′-second strand-apical loop sequence-first strand-optional 2 nt overhang sequence-3′. Another shRNA design may be a shRNA structure that is processed by the RNAi machinery to provide for an activated RISC complex that does not require Dicer processing (Liu et al., Nucleic Acids Res. 2013, Apr 1;41(6):3723-33, incorporated herein by reference), so called AgoshRNAs or Ago2 processed shRNAs, which are based on a structure very similar to the miR-451 scaffold as described below. Such a shRNA structure comprises in its loop sequence part of the first RNA sequence. Such a shRNA structure may also consist of the first strand, followed immediately by the second strand.

In one embodiment, there is provided a nucleic acid, wherein the sequence encoding the first RNA is followed by a first spacer and the sequence encoding the second RNA, wherein the spacer is at least 15 nucleotides. Thus, preferably, in a 5′ to 3′ direction, the sequence encoding the first RNA is followed by a first spacer comprising at least 15 nucleotides, which spacer is followed by the sequence encoding the second RNA. It is understood that the 5′ to 3′ direction refers to the coding strand in case of a double-stranded nucleic acid.

As described above, the nucleic acid may be said to derive, in a 5′ to 3′ direction, a first and second RNA strand, which is the first RNA, and a third and fourth RNA strand, which is the second RNA, wherein the sequences encoding the first and second RNA strands are followed by a first spacer of at least 15 nucleotides followed by the sequences encoding the third and fourth RNA strands. In some embodiments, the nucleic acid of the invention comprises a third sequence encoding a third RNA. In preferred embodiments of the invention, the sequence encoding the third RNA is followed by a second spacer comprising at least 15 nucleotides followed by the sequence encoding the first RNA. Thus, preferably, in a 5′ to 3′ direction, the sequence encoding the third RNA is followed by a second spacer comprising at least 15 nucleotides, which second spacer is followed by the sequence encoding the first RNA, followed by the first spacer and the sequence encoding the second RNA.

Thus, the nucleic acid may be said to derive a first and second RNA strand, which is the first RNA, a third and fourth RNA strand, which is the second RNA, and a fifth and second RNA strands, which is the third RNA, wherein, in a 5′ to 3′ direction, the sequences encoding the fifth and sixth RNA strands are followed by a spacer of at least 15 nucleotides and the sequences encoding the first and second RNA strands followed by a spacer of at least 15 nucleotides and the sequences encoding the third and fourth RNA strands.

It was fortuitously identified that the use of the spacer as described herein enables the downstream processing of the sequences encoding the first and second and further RNAs allowing their combinatorial effect. As above, said nucleic acid may be said to derive a first and second RNA strand, which is the first RNA, and a third and fourth RNA strand, which is the second RNA, and a fifth and second RNA strand, which is the third RNA, wherein the sequences encoding the first and second RNA strands are followed by a first spacer and the sequences encoding the third and fourth RNA strands, wherein the spacer is at least 15 nucleotides.

In some embodiments of the invention, the spacer comprises at least 25; at least 30; or at least 35 nucleotides. In some embodiments of the invention, the spacer comprises at least: 15; 20; 25; 30; 35; 40; 45; 50; 55; 60; 65; 70; 75; 80; 85; 90; 95; 100; 105; 110; 115; 120; 125; 130; 135; 140; 145; 150; 155; 160; 165; 170; 175; 180; 185; 190; 195; or 200 nucleotides. In some specific embodiments, the spacer of the invention comprises 15; 16; 17; 18; 19; 20; 21; 22; 23; 24; 25; 26; 27; 28; 29; 30; 31; 32; 33; 34; 35; 36; 37; 38; or 40 nucleotides. In some specific embodiments of the invention, the spacer comprises 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; or 105 nucleotides. In some embodiments of the invention, the spacer comprises or consists of a sequence selected from the group consisting of: SEQ ID NO:1; SEQ ID NO: 27 and SEQ ID NO: 116 and variants thereof.

In some embodiments of the invention, the sequences encoding the first and second RNA strands are followed by a spacer of at least 75 nucleotides and the sequences encoding the third and fourth RNA strands. In some specific examples of the invention, the sequences encoding the first and second RNA strands are followed by a spacer comprising or consisting of SEQ ID NO: 46.

In some embodiments of the invention, the sequences encoding the first and second RNA strands are followed by a spacer of at least 15 nucleotides and the sequences encoding the third and fourth RNA strands. In some specific examples of the invention, the sequences encoding the first and second RNA strands are followed by a spacer comprising or consisting of SEQ ID NO. 116.

In some embodiments of the invention, the sequences encoding the fifth and sixth RNA strands are followed by a spacer of at least 75 nucleotides and the sequences encoding the first and second RNA strands.
In some embodiments, the sequences encoding the fifth and sixth RNA strands are followed by a spacer of at least 75 nucleotides and the sequences encoding the first and second RNA strands are followed by a spacer of at least 15 nucleotides and the sequences encoding the third and fourth RNA strands. In some specific examples of the invention, the sequences encoding the fifth and sixth RNA strands are followed by a spacer comprising or consisting of SEQ ID NO. 1 and the sequences encoding the first and second RNA strands are followed by a spacer comprising or consisting of SEQ ID NO. 116 and the sequences encoding the third and fourth RNA strands.
In some embodiments of the invention, the first and second RNA comprise SEQ ID NO. 117 and the sequence encoding the first RNA is followed by a first spacer which comprises or consists of SEQ ID NO. 116.

In some embodiments of the invention, the first RNA comprises SEQ ID NO. 118 and the second RNA comprises SEQ ID NO. 117; and the sequence encoding the first RNA is followed by a first spacer which comprises or consists of SEQ ID NO. 1.

In some embodiments of the invention, the third RNA comprises SEQ ID NO. 119 and the first and second RNA comprise SEQ ID NO. 117 the sequence encoding the third RNA is followed by a second spacer which comprises or consists of SEQ ID NO. 1 and the sequence encoding the first RNA is followed by a first spacer which comprises or consists of SEQ ID NO. 116.
Thus, the first and second RNA may also be known as an RNA cluster, when transcribed from physically adjacent genes. In a further embodiment, in case one or both RNAs are shRNA to be processed by Dicer, the relevant above mentioned shRNA structures are also applicable. In an alternative embodiment, in case one or both RNAs are AgoshRNAs, the relevant above mentioned shRNA structures are also applicable. Thus, one or both RNAs may be processed by the same or different RNAi machinery, such as Dicer and/or Ago2of the RNAi machinery. In a preferred embodiment, the first RNA is processed by Dicer, therefore, the putative strands of the subsequent siRNA are linked via a stem loop sequence: 5′-first strand-apical loop sequence-second strand-optional 2 nt overhang sequence-3′ or, conversely, 5′-second strand-apical loop sequence-first strand-optional 2 nt overhang sequence-3′. As shown in the examples, the first and second strands of the invention, may be preferably incorporated in a pre-miRNA or a pri-miRNA scaffold derived from a pri-miRNA or pre-miRNA scaffold derived from miR-144 (SEQ ID NO. 2). In a preferred embodiment, the third and fourth strands of the invention are incorporated in a pri-miRNA or pre-miRNA scaffold derived from miR-451 (SEQ ID NO. 3). Thus the first and/or second RNA can be described as a hairpin or a double stranded RNA that is substantially complementary to itself. In a more preferred embodiment, the first RNA comprises SEQ ID NO. 2 (miR-144) or a variant thereof.

In some embodiments of the invention where the first RNA is incorporated in a pre-miRNA or a pri-miRNA scaffold derived from miR-144, the first RNA is mutated to reduce processing and/or expression of the first RNA. In some specific embodiments, SEQ ID NO. 4or the variant thereof is mutated to reduce processing and/or expression of the first RNA. In some embodiments, the mutation is a single point mutation. In other words, the first RNA comprises a single point mutation to reduce processing and/or expression of the first RNA. In some preferred embodiments, the single point mutation is A-T-position 19 (SEQ ID NO. 4). The skilled person can easily determine whether this is the case by using standard assays and appropriate controls such as described in the examples and as known in the art.

Thus, in some embodiments, the first RNA is processed by Dicer and the second RNA is processed by Ago2, such as an AgoshRNA, or miR-451 mimic RNA and may be said to comprise, in its loop sequence, part of the second RNA sequence. Such a shRNA structure may also consist of the third strand, followed immediately by the fourth strand of the invention.

A double stranded RNA according to the invention may also be incorporated in a pre-miRNA or pri-miRNA scaffold. MicroRNAs, i.e. miRNA, are guide strands that originate from double stranded RNA molecules that are endogenously expressed e.g. in mammalian cells. Two or more miRNAs may also be comprised in a miRNA cluster, wherein the miRNAs in the cluster are transcribed in the same orientation and are not separated by a transcription unit or a miRNA in the opposite orientation. A miRNA is processed from a pre-miRNA precursor molecule, similar to the processing of a shRNA or an extended siRNA as described above, by the RNAi machinery and incorporated in an activated RNA-induced silencing complex (RISC) (Tijsterman M, Plasterk RH. Dicers at RISC; the mechanism of RNAi. Cell. 2004 Apr. 2; 1 17(1):1-3). A pre-miRNA is a hairpin RNA molecule that can be part of a larger RNA molecule (pri-miRNA), e.g. comprised in an intron, which is first processed by Drosha to form a pre-miRNA hairpin molecule. In one embodiment, the hairpin in the second RNA comprises at least 40 nucleotides.

Said third RNA may be incorporated in a pre-miRNA or a pri-miRNA scaffold derived from miR-144. In some specific embodiments of the invention, the third RNA comprises SEQ ID NO. 119 or a variant thereof. In other words, and as described above, the first and second strands of the third RNA of the invention are incorporated in a pre-miRNA or a pri-miRNA scaffold derived from miR-144. Thus, the third RNA can be described as a hairpin or a double stranded RNA that is substantially complementary to itself. In some embodiments, the hairpin in the second RNA comprises at least 70 nucleotides.

In some specific embodiments, the nucleic acid of the invention comprises a first, a second and a third RNA, wherein the first and second RNA are incorporated in a pre-miRNA or a pri-miRNA scaffold derived from miR-451 and the third RNA is incorporated in a pre-miRNA or a pri-miRNA scaffold derived from miR-144. In some specific embodiments, the first and second RNA comprises SEQ ID NO. 117 or a variant thereof and the third RNA comprises SEQ ID NO. 119 or a variant thereof. The variants thereof comprise SEQ ID NO. 120.

In said embodiments where the third RNA is incorporated in a pre-miRNA or a pri-miRNA scaffold derived from miR-144, the third RNA is processed by Dicer; therefore, the putative strands of the subsequent siRNA are linked via a stem loop sequence: 5′-first strand-apical loop sequence-second strand-optional 2 nt overhang sequence-3′ or, conversely, 5′-second strand-apical loop sequence-first strand-optional 2 nt overhang sequence-3′.

In some embodiments, the nucleic acid comprising a sequence encoding a first RNA and a sequence encoding a second RNA further comprises a sequence encoding a third RNA, wherein the third RNA comprises SEQ ID NO; 119 or a variant thereof.

In some embodiments of the invention where the third RNA is incorporated in a pre-miRNA or a pri-miRNA scaffold derived from miR-144, the third RNA is preferably mutated to reduce processing and/or expression of the third RNA. In some specific embodiments, SEQ ID NO. 119 or the variant thereof is mutated to reduce processing and/or expression of the third RNA.

In some embodiments, the mutation is a single point mutation. In other words, the third RNA comprises a single point mutation to reduce processing and/or expression of the third RNA. Any mismatch, bulge or GU wobble introduced within positions 4-8 of the DROSHA cleavage site may impair the enzymatic activity of DROSHA. Double and triple mismatches, bulges or wobbles within said positions further decrease the activity of DROSHA. Therefore, any of the following single nucleotide polymorphisms (SNPs) and combinations thereof within the 4-8 nucleotide stretch of miR-144 may alter (pre-)-miR-144 expression.

In some embodiments, the third RNA comprises at least one mutation selected from the group consisting of: U>G at position 4; A>U or G at position 5; U>A at position 6; C>G or U at position 7; and A>U or G at position 8.

In preferred embodiments, the third RNA comprises: a single point mutation A>U at position 5. The skilled person can easily determine whether this is the case by using standard assays and appropriate controls such as described in the examples and as known in the art.

The specific combination of miR-144/miR-451 scaffolds, specifically those comprising SEQ ID NO. 119 or SEQ ID NO. 120 and those comprising SEQ ID NO. 117, within the nucleic acid of the invention is particularly helpful within the context of gene therapy and RNA silencing. miR-144 and miR-451 are examples of clustered miRNAs regulated in trans, wherein miR-144 regulates the processing of miR-451 by Ago2. Specifically, miR-144 enhances miR-451 biogenesis in trans by repressing Dicer and, in turn, repressing global canonical miRNA processing (Kretov et al., 2020, Molecular Cell 78, 317-328). Thus, where this combination of scaffolds is used, the third RNA of the invention plays a key role in enhancing the biogenesis of the first and second RNA of the invention, and therefore, the delivery of the guide sequences comprised in said first and second RNAs.

In some embodiments of the invention, the first RNA is incorporated into a miR-144 scaffold.

In some specific embodiments of the invention, the first RNA is incorporated into a miR-144 scaffold, and the second RNA is incorporated into a miR-451 scaffold.

As stated above, the first RNA comprises a first guide sequence. In those embodiments of the invention where the first RNA is incorporated into a miR-144 scaffold, the first guide sequence may be incorporated into the first or the second strand of the first RNA.

In those embodiments where the first RNA of the invention is incorporated into a miR-144 scaffold, the miRNA 144 scaffold for use in the invention preferably comprises from 5′ to 3′, firstly 5′-TGGGGCCCTGGC™-3′ (wherein M is an A or a C or a G or a U) (SEQ ID NO. 121), followed by a sequence of 22 nucleotides, comprising or consisting of a first RNA sequence, followed by a sequence of 15 nucleotides which can be regarded as the apical loop 5′-TTTGCGATGAGAWMM-3′ (wherein W is preferably a C or a G, but can also be a A or a U, and is to base pair with the last nucleotide of the first strand of the first RNA; and wherein M is an A or a C and is not to base pair with nucleotide 21 of the first strand of the first RNA) (SEQ ID NO. 122), followed by a sequence of 20 nucleotides, which can be regarded as the second RNA sequence, and which is complementary over its entire length with nucleotides 1 and 3-10 and 12-20 of said sequence of 22 nucleotides, except nucleotide 18 that forms a mismatch with nucleotide 2 of the first strand, subsequently followed by sequence 5′-AGTCCGGGCACCCCC-3′ (SEQ ID NO. 123).

Such a scaffold may comprise flanking sequences as found in the original pri-miR-144 scaffold. Alternatively, the flanking sequences 5′-ATCGGCGCTATGCTTCCTGTGCCCCCAG-3′ (SEQ ID NO. 124) and 5′-AGCTCTGGAGCCTGACAAGGAGGACAGGAGAGATGCTGCAAGCCCAAGAAGCTCTCTGCTCAGCCT GTCACAACCTACTGACTGCCAGGGCA-3′ (SEQ ID NO. 1 may be replaced by flanking sequences of other pri-mRNA structures.

In those embodiments where the first RNA of the invention comprises a miR-144 scaffold, the guide sequence may be comprised within the 5p- or 3p-arm of the scaffold. DROSHA processing of miR-144, subsequently followed by DICER processing, generate a miRNA duplex which ultimately enters the process of miRNA strand selection. Strand selection is operated within the RISC and determines which strand will become the active strand (also referred to as the guide strand) and which strand will be degraded (passenger strand) (Noland and Doudna, 2013 RNA, 19:639-648). Guide strand selection is highly determined by thermodynamic characteristics of the miRNA duplex, and thus it may be influenced by modifying the nucleotide sequence encoding the miRNA. Generally speaking, the strand with the lower thermodynamic stability at its 5′ end will become the guide strand. Another key feature of human miRNA guide strands is a 5′ end U-bias associated with an enrichment for A and G nucleotides, whilst the passenger strands exhibit a 5′ end C-bias and an enrichment for C and U nucleotides (Hai Yang Hu et al. 2009, BMC Genomics 2009, 10:413).

Conveniently, since the guide strand selection is sequence dependent, the miR-144 constructs of the invention comprising guide strands may be designed as 5p- or 3p-guide-containing regions (SEQ ID NO 121). Importantly, the probability with which the desired guide strand of the invention will be selected in RISC can be modulated by single nucleotide variations such as selecting U as the first nucleotide of the first strand (5p-design) or the first nucleotide of the second strand (3p-designs) and selecting C or G as nucleotide 20 of the first strand (5p-designs) or nucleotide 19 of the second strand (3p-designs).

As with miR-451 scaffolds, the sequence of the miR-144 scaffold may differ not only with regard to the (putative) guide strand sequence, and sequence complementary thereto, as present in the wild-type scaffold, but may also comprise additional mutations in the 5′, loop and 3′ sequence as well, as additional mutations may be required to provide for an RNA structure that is predicted to mimic the secondary structure of the wild-type scaffold. Again, such a scaffold may be comprised in a larger RNA transcript, e.g. a pol II expressed transcript, comprising e.g. a 5′ UTR and a 3′UTR and a poly A. Flanking structures may also be absent. An expression cassette in accordance with the invention may thus express a shRNA-like structure having a sequence of 22 nucleotides, comprising or consisting of the first strand of the first RNA, followed a sequence of 20 nucleotides, which can be regarded to be the second strand of the first RNA, which is complementary over its entire length with nucleotides 1 and 3-10 and 11-19 of said sequence of 22 nucleotides, except nucleotide 18 that forms a mismatch with nucleotide 2 of the first strand. The latter shRNA-like structure derived from the miR-144 scaffold can be referred to as a pre-miRNA scaffold from miR-144. Alternatively, the flanking sequences, 5′-ATCGGCGCTATGCTTCCTGTGCCCCCAG-3′ and 5′-AGCTCTGGAGCCTGACAAGGAGGACAGGAGAGATGCTGCAAGCCCAAGAAGCTCTCTGCTCAGCCT GTCACAACCTACTGACTGCCAGGGCA-3′ (SEQ ID NO: 124 and SEQ ID NO:1, respectively) may be replaced by flanking sequences of other pri-mRNA structures. Flanking structures may also be absent.

In some preferred embodiments, the first or the second strand of the first RNA has a length of 19, 20, 21, or 22 nucleotides. In some specific embodiments, the first or second strand of the first RNA is fully complementary over its entire length with the first target sequence. In some preferred embodiments, the first or the second strand of the first RNA has a length of 19, 20, 21, or 22 nucleotides, wherein said first strand of the first RNA is fully complementary over its entire length with the target sequence.

As explained above, where the first strand of the first RNA comprises a guide sequence, the first strand is to be combined with a second strand of the first RNA. As above, the skilled person is well capable of designing and selecting a suitable second strand of the first RNA.

Similarly, where the second strand of the first RNA comprises a guide sequence, the second strand is to be combined with a first strand of the first RNA. As above, the skilled person is well capable of designing and selecting a suitable first strand of the first RNA A suitable scaffold comprising a first and second strand for the first RNA in accordance with the invention can be a sequence such as SEQ ID NO. 118.

Thus, in some embodiments of the invention, the first RNA comprises SEQ ID NO. 118 or a variant thereof. In some more specific embodiments of the invention, the first RNA comprises SEQ ID NO. 118 or a variant thereof; and the second RNA comprises SEQ ID NO. 117 or a variant thereof.

Hence, where the first RNA comprises SEQ ID NO. 118, the first guide sequence may be incorporated into the first or the second strand of the first RNA.

The first and/or second RNA to be expressed in accordance with the invention comprise, in whole or a substantial part thereof, a guide strand.

In certain embodiments of the invention, the guide strand may also be referred to as antisense strand as it is complementary (“anti”) to a target RNA sequence in a sense or antisense transcript, the sense or antisense target RNA sequence being comprised in an RNA encoded by a gene of choice. Thus, the first and/or second RNA also comprise a “sense strand”, that may have substantial sequence identity with, or be identical to, the target RNA sequence. Therefore, the first and/or second RNA can be described as a hairpin or a double stranded RNA that is substantially complementary to itself.

Said double stranded RNA according to the invention is to induce RNA interference, thereby reducing expression of transcripts encoded by the gene of choice, which includes knocking down of said transcripts. Transcripts that may be targeted may include spliced, including splice variants, and unspliced RNA transcripts. Thus, an RNA encoded by a gene of choice is understood to comprise unspliced mRNAs comprising a 5′ untranslated region (UTR), intron and exon sequences, followed by a 3′ UTR and a polyA signal, and also splice variants thereof. The double stranded RNA according to the invention may also induce transcriptional silencing.

Reducing expression of a transcript encoded by the gene of choice is herein thus preferably understood as reducing the steady state level of a functional mRNA encoded by the gene of choice in a target cell such that less of the mRNA is available in the cell for translation into the product encoded by the gene of choice, thereby reducing the steady state level of the protein in the target cell. Reducing expression of a transcript encoded by the gene of choice therefore does not necessarily involve reducing de novo transcription of the gene but rather increased degradation of an mRNA encoded by the gene of choice and/or its precursors, e.g. unspliced RNA transcripts.

In some embodiments, the double stranded RNA according to the invention comprises a first RNA sequence and a second RNA sequence, wherein the first and second RNA sequences are substantially complementary, and wherein the first RNA sequence has a sequence length of at least 19 nucleotides and is substantially complementary to a target sequence in a transcript encoded by a first or second gene, which first RNA sequence is capable of inducing RNA interference to sequence-specifically reduce expression of an RNA transcript comprising the target RNA sequence. In some further embodiments, said induction of RNA interference to reduce expression of an RNA transcript comprising the target RNA sequence means that it is to reduce the first and second gene expression.

Similarly, in some embodiments, the double stranded RNA according to the invention comprises a first RNA sequence and a second RNA sequence, wherein the first and second RNA sequences are substantially complementary, and wherein the second RNA sequence has a sequence length of at least 19 nucleotides and is substantially complementary to a target sequence in a transcript encoded by a first or second gene, which first RNA sequence is capable of inducing RNA interference to sequence-specifically reduce expression of an RNA transcript comprising the target RNA sequence. In some further embodiments, said induction of RNA interference to reduce expression of an RNA transcript comprising the target RNA sequence means that it is to reduce the first and second gene expression.

The skilled person can easily determine whether this is the case by using standard luciferase reporter assays and appropriate controls such as described in the examples and as known in the art (Zhuang et al. 2006 Methods Mol Biol.342:181-7). For example, a luciferase reporter comprising a target RNA sequence can be used to show that the double stranded RNA according to the invention is capable of sequence specific knock down. Furthermore, levels of gene expression can be determined by detecting endogenous mRNA levels (nuclear and/or cytoplasmic), RNA foci formation, and endogenous protein levels.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

It is understood that “substantially complementary” in this context means that it is not required to have all the nucleotides of the guide sequence and the target sequence to be base paired, i.e. to be fully complementary, or all the nucleotides of the guide sequence and the target sequence to be base paired. As long as the first and/or second RNAs of the invention are capable of inducing RNA interference to thereby sequence-specifically target a sequence comprising the target RNA sequence, such substantial complementarity is contemplated in accordance with the invention.

The substantial complementarity between the strand complimentary to the target RNA sequence, which may also be referred to as part of a first or second gene, preferably consists of at most two mismatched nucleotides, more preferably having one mismatched nucleotide, most preferably having no mismatches. It is understood that one mismatched nucleotide means that over the entire length of the strand complimentary to the target RNA sequence when base paired with the target RNA sequence one nucleotide does not base pair with the target RNA sequence. Having no mismatches means that all nucleotides of the strand complimentary to the target RNA base pair with the target RNA sequence, having 2 mismatches means two nucleotides of the strand complimentary to the target RNA do not base pair with the target RNA sequence. The strand complimentary to the target RNA may also comprise additional nucleotides that do not have complementarity to the target RNA sequence, and may be longer than e.g. 21 nucleotides. In such a scenario, the substantial complementarity is determined over the entire length of the target RNA sequence. This means that the target RNA sequence in this embodiment has either no, one or two mismatches over its entire length when base paired with the strand complimentary to the target RNA.

Having a few mismatches between the strand complimentary to the target RNA and the target RNA sequence may be allowed according to the invention, as long as the double stranded RNA according to the invention is capable of reducing expression of transcripts comprising the target RNA sequence, such as a luciferase reporter or e.g. a transcript comprising the target RNA sequence. In said embodiments, substantial complementarity between the strand complimentary to the target RNA and the target RNA sequence consists of having no, one or two mismatches over the entire length of either the strand complimentary to the target RNA or the target RNA sequence encoded by an RNA of a first or second human gene, whichever is the shortest.

In certain embodiments, the first RNA comprises a first guide sequence and second RNA comprises a second guide sequence, wherein both guide sequences are substantially complementary to a first and a second target sequence in a first and second transcript encoded by a first and second gene.

In certain embodiments, said first and second gene are the same gene.

In certain embodiments, said first and second target sequences are the same.

In certain embodiments, said first and second target sequences are different target sequences.

In certain embodiments, said first and second gene are different genes.

As said, a mismatch according to the invention means that a nucleotide of the first (or second) and third strand (the strand) does not base pair with the target RNA sequence encoded by an RNA of the first or second gene. Nucleotides that do not base pair are A and A, G and G, C and C, U and U, A and C, C and U, or A and G. A mismatch may also result from a deletion of a nucleotide, or an insertion of a nucleotide. When the mismatch is a deletion in the strand sequence, this means that a nucleotide of the target RNA sequence is not base paired with the sequence when compared with the entire length of the strand sequence. Nucleotides that can base pair are A-U, G-C and G-U. A G-U base pair is also referred to as a G-U wobble, or wobble base pair. In one embodiment the number of G-U base pairs between the strand sequence and the target RNA sequence is 0, 1 or 2. In some embodiments, there are no mismatches between the strand RNA sequence and the target RNA sequence and a G-U base pair or G-U pairs are allowed. Preferably, there may be no G-U base pairs between the strand sequence and the target RNA sequence, or the strand sequence and the target RNA sequence only have base pairs that are A-U or G-C. Preferably, there are no G-U base pairs and no mismatches between the strand sequence and the target RNA sequence. The strand sequence of the double stranded RNA according to invention preferably is fully complementary to the target RNA sequence, said complementarity consisting of G-U, G-C and A-U base pairs. The strand sequence of the double stranded RNA according to invention more preferably is fully complementary to the target RNA sequence, said complementarity consisting of G-C and A-U base pairs.

Therefore, in some embodiments, the strand sequence and the target RNA sequence have at least 15, 16, 17, 18, or 19 nucleotides that base pair. Preferably the strand and the target RNA sequence are substantially complementary, said complementarity comprising at least 19 base pairs. In other embodiments, the strand has at least 8, 9, 10, 11, 12, 13 or 14 consecutive nucleotides that base pair with consecutive nucleotides of the target RNA sequence. In other embodiments, the strand has at least 19 consecutive nucleotides that base pair with consecutive nucleotides of the target RNA sequence. In other embodiments, the strand comprises at least 19 consecutive nucleotides that base pair with 19 consecutive nucleotides of the target RNA sequence. In other embodiments, the strand has at least 17 nucleotides that base pair with the target RNA sequence and have at least 15 consecutive nucleotides that base pair with consecutive nucleotides of the target RNA sequence. The sequence length of the first strand is preferably at most 21, 22, 23, 24, 25, 26, or 27 nucleotides. In other embodiments, the strand has at least 20 consecutive nucleotides that base pair with 20 consecutive nucleotides of the target RNA sequence. In other embodiments, the strand comprises at least 21 consecutive nucleotides that base pair with 21 consecutive nucleotides of the target RNA sequence.

As said, it may be not required to have full complementarity (i.e. full base pairing (no mismatches) and no G-U base pairs) between the first or second strand of the first RNA and the first strand of the second RNA, and the target RNA sequence as such a strand can still allow for sufficient suppression of gene expression. Also, not having full complementarity may be contemplated for example to avoid or reduce off-target RNA sequence specific gene suppression while maintaining sequence specific inhibition of transcripts comprising the target RNA sequence. However, it may be preferred to have full complementarity as it may result in more potent inhibition. Without being bound by theory, having full complementarity between the first or second strand of the first RNA and/or the first strand of the second RNA, and the target RNA sequence may allow for the activated RISC complex comprising said first or second strand of the first RNA or the first strand of the second RNA (or a substantial part thereof) to cleave its target RNA sequence, whereas having mismatches may hamper cleavage and can result in mainly allowing inhibition of translation, of which the latter may result in less potent inhibition.

With regard to the second strand on the RNA of the invention, the second strand is substantially complementary with the first strand on the RNA. The second strand combined with the first strand forms a double stranded RNA. As said, this is to form a suitable substrate for the RNA interference machinery such that a guide sequence derived from the first strand is comprised in the RISC complex in order to sequence specifically inhibit expression of its target, i.e. RNA encoded by the first or second gene. The sequence of the second strand has sequence similarity with the target RNA sequence. However, the substantial complementarity of the second strand with the first strand may be selected to have less substantial complementarity as compared with the substantial complementarity between the first strand and the target RNA sequence. Hence, the second strand may comprise 0, 1, 2, 3, 4, or more mismatches, 0, 1, 2, 3, or more GU wobble base pairs, and may comprise insertions of 0, 1, 2, 3, 4, nucleotides and/or deletions of 0, 1, 2, 3, 4, nucleotides. It is understood that, where the guide sequence is comprised within the second strand of the first RNA, or the second strand of the invention, the description above applies to the first strand of the invention.

Preferably the first strand and the second strand are substantially complementary, said complementarity comprising 0, 1, 2 or 3 G U base pairs and/or wherein said complementarity comprises at least 17 base pairs. These mismatches, G-U wobble base pairs, insertions and deletions, are with regard to the first strand, i.e. the double stranded region that is formed between the first and second strands. As long as the first and second strands can substantially base pair, and are capable of inducing sequence specific inhibition of an RNA encoded by the first or second gene, such substantial complementarity is allowed according to the invention. It is also understood that substantially complementarity between the first and the second strands may depend on the double stranded RNA design of choice. It may depend for example on the miRNA scaffold that is chosen for in which the double stranded RNA is to be incorporated. As is clear from the above, the substantial complementarity between the first strand and second strand of the first and/or second RNA may comprise mismatches, deletions and/or insertions relative to a first and second RNA sequence being fully complementary (i.e. fully base paired). In some embodiments, the first and second strands of the first and/or second RNA have at least 11 consecutive base pairs. Hence, at least 11 consecutive nucleotides of the first strand and at least 11 consecutive nucleotides of the second strand are fully complementary. In some embodiment the first and second strands of the first and/or second RNA have at least 15 nucleotides that base pair. Said base pairing between at least 15 nucleotides of the first strand and at least 15 nucleotides of the second strand may consist of G-U, G-C and A-U base pairs, or may consist of G-C and A-U base pairs.

In some embodiments, the first and second RNA sequences have at least 15 nucleotides that base pair and have at least 11 consecutive base pairs. In another embodiments, the first RNA sequence and the second RNA sequence are substantially complementary, wherein said complementarity comprises at least 17 base pairs. Said 17 base pairs may preferably be 17 consecutive base pairs, said base pairing consisting of G-U, G-C and A-U base pairs or consisting of G-C and A-U base pairs.

The invention thus provides for an expression cassette encoding the first strand and second strand of the first and second RNA wherein the first and second strands are substantially complementary, wherein the first or second strand of the first RNA and the first strand of the second RNA has a sequence length of at least 19 nucleotides and is substantially complementary to a target RNA sequence comprised in an RNA encoded by the first or second gene.

In some embodiments, an expression cassette is provided encoding a first strand and a second strand wherein the first and second strands of the first and second RNA are substantially complementary, wherein the first strand or second strand of the first RNA and the first strand of the second RNA has a sequence length of at least 22 nucleotides and is substantially complementary to a target RNA sequence comprised in an RNA encoded by the first or second gene. Where applicable, the expression cassette may also encode the first strand and second strand of the third RNA, wherein the first and second strands are substantially complementary.

As stated above, the RNAs of the invention may be incorporated into miRNA scaffolds. The miRNA scaffold sequence is processed by the RNAi machinery as present in the cell. In some specific embodiments of the invention, the processing of the miRNA scaffold sequence results in: guide sequences comprising the first strand of the first RNA, or a substantial part thereof, in the range of 21-30 nucleotides; and guide sequences comprising the first strand of the second RNA, or a substantial part thereof, in the range of 21-30 nucleotides. In some other specific embodiments of the invention, the processing of the miRNA scaffold sequence results in: guide sequences comprising the second strand of the first RNA, or a substantial part thereof, in the range of 21-30 nucleotides; and guide sequences comprising the first strand of the second RNA, or a substantial part thereof, in the range of 21-30 nucleotides. Such guide strands are capable of reducing the first and second gene transcript expression by targeting the selected target sequences.

The first strand of the first RNA and the first strand of the second RNA, and/or, alternatively, the second strand of the first RNA and the first strand of the second RNA, as it is encoded by the expression cassette of the invention, is comprised in part or in whole, in a guide strand when it has been processed by the RNAi machinery of the cell. Hence, the guide strand that is to be generated from the RNA encoded by the expression cassette, comprising the first or second strand of the first RNA and the first strand of the second RNA, is to comprise at least 18 nucleotides of the second RNA sequence.

Preferably, such a guide strand comprises at least 19 nucleotides, 20 nucleotides, 21 nucleotides, or 22 nucleotides. A guide strand can comprise the first or second strand of the first or the first strand of the second RNA sequence, also as a whole. In selecting a miRNA scaffold, the first or second strand of the first RNA sequence and the first strand in the second RNA sequence, can be selected such that it is to replace the original guide strand. However, this does not necessarily mean that the guide strand produced from such an artificial scaffold is identical in length to the first or second strand of the first RNA or the first strand of the second RNA selected, nor that the first or second strand of the first RNA or the first strand of the second RNA is in its entirety to be found in the guide strand that is produced.

In one aspect, the invention relates to a kit comprising the gene delivery vehicle of the invention, or the pharmaceutical composition according to the invention, and an immunosuppressive compound.

In another aspect, the invention relates to a cell comprising the gene delivery vehicle according to the invention, or a host cell.

In one embodiment, the expression cassette is a DNA molecule. Such DNA molecules may be useful in the application of further technologies and applications, providing a vector for the nucleic acids as described herein.

The gene delivery vehicle according to the invention further comprises a sequence encoding a full length antibody or fragment thereof. Thus the gene delivery vehicle according to the invention comprises a nucleic acid sequence encoding a heavy chain and a light chain of an antibody. The heavy chain and light
chain are expressed and assembled to form the antibody which is secreted. As used herein, the term “antibody” is referred to in the broadest sense and specifically covers various embodiments including but not limited to monoclonal antibodies (mAbs), polyclonal antibodies, multispecific antibodies (e.g., bispecific antibodies formed from at least two intact antibodies), and antibody fragments (e.g., diabodies) so long as they exhibit a desired biological activity (e.g., “functional”). Examples of antibody fragments or mAb-derived binding fragments may include, but are not limited to Fab, Fab′, F (ab′) 2, scFv and Fv fragments; diabodies, linear antibodies, single-chain antibody molecules; and multispecific antibodies formed from antibody fragments. Papain digestion of antibodies produces two identical antigen-binding fragments, called “Fab” fragments, each with a single antigen binding site. In one embodiment, a mAb or binding fragment thereof according to the invention is not comprised within a chimeric antigen receptor (CAR) or monoclonal T Cell Receptor (mTCR).

In certain embodiments, the gene delivery vehicle according to the invention comprises antibodies that are known in the art and/or antibodies that are commercially available. Further, polypeptides of the invention may include fragments of such antibodies or antibodies that have been developed to comprise one or more of such fragments (e. g. variable domains or complementarity determining regions (CDRs). In some embodiments, viral genomes of the AAV particles of the invention may encode antibodies or antibody-based compositions produced using methods known in the art. In certain embodiments, the gene delivery system encodes antibody PRX002 as described for example in Schenk, et al. Movement disorders 32.2 (2017): 211-218 9—which is herein incorporated by reference. In certain embodiments, the gene delivery system encodes antibody MED11341 as described for example in Schofield et al, Neurobiology of disease 132 (2019): 104582.—which is herein incorporated by reference.

The terms “expression vector” or “expression cassette” refer to nucleotide sequences that are capable of effecting expression of a gene in host cells or host organisms compatible with such sequences. As is known in the art, expression of a gene product requires the presence of expression control and/or regulatory sequences such as one or more promoters and any other nucleic acid sequences, such as introns, necessary for expression of the selected nucleic acid sequence, all operably linked to the selected sequence, and may include an enhancer sequence. Thus, in one embodiment, the expression cassette comprising the at least one nucleic acid as described herein and encoding the full-length mAb or an antigen binding fragment thereof as described herein comprises at least one mammalian cell-compatible expression control sequence, e.g. a promoter, that is/are operably linked to the nucleic acid and/or antibody, thus forming an expression cassette for expression of the nucleic acid and/or antibody the mammalian target cell to be treated by gene therapy. Many such promoters are known in the art (see Sambrook and Russel, 2001, supra). Constitutive promoters that are broadly expressed in many cell types, such as the CMV promoter may be used. However, more preferred will be promoters that are inducible, tissue-specific, cell-type-specific, or cell cycle-specific. Preferably a pol II promoter is used, such as a CAG promoter (SEQ ID NO. 5) (i.a. Miyazaki et al. Gene. 79 (2): 269-77; Niwa, Gene. 108 (2): 193-9), a phosphoglycerate kinase (PGK) promoter (Hannan et al. Gene. 130:233-9), a CMV promoter (Such as depicted e.g. in FIG. 2 of WO2016102664, which is herein incorporated by reference) an adapted/synthetic promoter (P1, SEQ ID NO. 192 and P2, SEQ ID NO. 193), or a truncated chicken β-actin (CBA) hybrid (CBh) promoter (Gray et al. Human Gene Therapy. 22 (9): 1143-1153). In some preferred embodiments the promoter is a neuro specific promoter. Thus, in some embodiment, the promoter is a promoter capable of driving transcription in a brain cell. Examples of suitable neuro specific promoters are Neuron-Specific Enolase (NSE), human synapsin 1, caMK kinase and tubulin (Hioki et al. Gene Ther. 2007 June;14(11):872-82). Other suitable promoters that can be contemplated are inducible promoters, i.e. a promoter that initiates transcription only when the host cell is exposed to some particular stimulus.

The expression cassette for expression of at least one nucleic acid and an antibody, as described above, further preferably encodes a polyA signal comprised in the DNA expression cassette operably linked to the 3′ end of the RNA molecule. Preferably, said polyA signal is the simian virus 40 polyadenylations (SV40 polyA, SEQ ID NO. 8), a synthetic polyadenylation signal, the Bovine Growth Hormone polyadenylation signal (BGH polyA), or the Human Growth Hormone polyadenylation signal (HGH polyA).

As explained herein, the gene delivery vehicle of the invention comprises at least one nucleic acid encoding an RNA that comprises a guide sequence that is substantially complementary to a target sequence in a gene associated with a disease and encodes an antibody of antigen binding fragment thereof that is targeting a protein associated with that same disease. The gene delivery system as described herein is thus capable targeting a disease intracellularly (by lowering mRNA levels in the cell through the use of miRNAs) and extracellularly (by targeting the proteins secreted by the cell) at the same time.

It is understood that “substantially complementary” in this context means that it is not required to have all the nucleotides of the guide sequence and the target sequence to be base paired, i.e. to be fully complementary, or all the nucleotides of the guide sequence and the target sequence to be base paired. As long as the second RNA is capable of inducing RNA interference to thereby sequence-specifically target a sequence comprising the target RNA sequence, such substantial complementarity is contemplated in accordance with the invention.

In certain embodiments, the nucleic acid comprised within the gene delivery vehicle of the invention comprising a first and a second RNA encodes for at least one further RNA. Preferably said further RNA comprising a further guide sequence. The guide sequence of the further RNA may be identical to the guide sequence of the second RNA as described however the further guide sequence may also differ from the guide sequence of the second RNA as long as the guide sequences target genes that are associated with the same disease.

As described in the first aspect of the invention, the gene delivery vehicle comprises an expression cassette that comprises at least one nucleic acid and that encodes a full length antibody or fragment thereof. In other words, the expression cassette enables the expression of the first, second (and optionally further) RNA and antibody or fragment thereof from a single construct.

The use of a combination of gene delivery vehicles, thereby providing for the use of multiple constructs (i.e. one gene delivery comprising the nucleic acid encoding the first and second and optionally third RNA and a separate gene delivery vehicle encoding the full length antibody or fragment thereof) is provided herein. Accordingly, in a second aspect, the invention provides for a combination of gene delivery vehicles wherein a first gene delivery vehicle comprises a first expression cassette comprising at least one nucleic acid comprising a sequence encoding a first RNA and a sequence encoding a second RNA and optionally a sequence encoding a third RNA, wherein the second RNA and optionally the first RNA comprise a guide sequence and wherein the first and second and optionally third RNA each comprise a hairpin and a second delivery vehicle comprising a second expression cassette, comprising a transgene encoding a full-length mAb or an antigen binding fragment thereof, wherein the guide sequence is substantially complementary to a target sequence in a gene associated with a disease and wherein the mAb or antigen binding fragment thereof is targeting a protein associated with that same disease. An exemplary list of target sequences and proteins causing the same disease is provided in Table 1.

In certain embodiments, the combination of the second aspect of the invention further comprises at least one further gene delivery vehicle comprising at least one further expression cassette comprising at least one further nucleic acid sequence encoding a further RNA. Preferably, this further RNA comprises a further guide sequence. The guide sequence of the further RNA may be identical to the guide sequence of the second RNA as described however the further guide sequence may also differ from the guide sequence of the second RNA as long as the guide sequences target genes that are associated with the same disease.

In certain embodiments, the gene delivery vehicle according to the first aspect of the invention or the combination according to the invention comprises a second RNA that comprises a guide sequence that is substantially complementary to a target sequence in a gene that encodes the antigen bound by the full-length mAb or an antigen binding fragment thereof. For example, in certain embodiments of the invention, the second RNA sequence comprises a guide sequence that is substantially complementary to a part of an alpha-synuclein (SNCA) gene while the antibody is an anti-alpha-synuclein antibody.

In certain embodiments, the gene delivery vehicle as described herein is viral vector. In preferred embodiments, the viral vector is AAV. Therefore, in some embodiment, an expression cassette as disclosed herein is flanked by Inverted Terminal Repeats. Preferably the AAV vector that is used is an AAV vector of serotype 5 or of serotype 9. AAV of serotype 5 (also referred to as AAV5) may be particularly useful for transducing human neurons and human astrocytes. Thus, AAV5 can efficiently transduce different human cell types of the CNS including FBN, dopaminergic neurons, motor neurons and astrocytes and is therefore a suitable vector candidate to deliver therapeutic genes to the CNS to treat neurogenerative diseases. The production of AAV vectors comprising any expression cassette of interest is well described in; WO2007/046703, WO2007/148971, WO2009/014445, WO2009/104964, WO2011/122950, WO2013/036118, which are incorporated herein in its entirety.

AAV sequences that may be used in the present invention for the production of AAV vectors, e.g. produced in insect or mammalian cell lines, can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 are preferred sources of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries. AAV capsids may consist of VP1, VP2 and VP3, but may also consist of VP1 and VP3. In some embodiment, the AAV vector according to the inventions comprises AAV5 or AAV9 capsid protein. In some embodiment, the AAV vector according to the inventions comprises AAV5 capsid protein. In some embodiment, the AAV vector according to the inventions comprises AAV9 capsid protein.

In another embodiment, a host cell is provided comprising the said nucleic acid or said expression cassette according to the invention. For example, the said expression cassette or nucleic acid may be comprised in a plasmid contained in bacteria. Said expression cassette or nucleic acid may also be comprised in a production cell that produces e.g. a viral vector. Said expression cassette may also be provided in a baculovirus vector.

Various modifications of the nucleotide sequences as defined above, including e.g. the wildtype AAV sequences, for proper expression in the host cell is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions are known to the skilled artisan which could increase the yield of the encoded proteins. These modifications are within the scope of the present invention.

In certain embodiments, any mammalian cell may be infected by an AAV vector of the invention. In a preferred embodiment, the cell to be infected is a human cell.

In some embodiments, the ITRs and capsid proteins (or parts thereof) in the AAV vector of the invention may be from different AAV serotypes. By way of example, and not limitation, the ITRs may be derived from AAV2, whilst the capsid proteins may be derived from a different serotype, for example AAV5 or AAV9.

In certain embodiments, the invention provides a composition comprising an AAV vector according to the invention and suitable excipients, such as buffers and stabilizers, antioxidants etc. In one particular embodiment, these compositions are used to transduce cells in vitro or ex vivo, in which case the excipients will need to be compatible with cell culture.

In a further aspect of the invention, the invention provides for a pharmaceutical composition comprising the gene delivery vehicle of the first aspect of the invention or the combination according to the second aspect of the invention and at least one pharmaceutically acceptable excipient. A pharmaceutical composition typically comprise physiological buffers, such as e.g. PBS, comprising further stabilizing agents such as e.g. sucrose. Such compositions are compatible with and suitable and intended for use in subsequent intravenous, intra-CSF (intrathecal, intra cisterna magna or intracerebroventricular), intraparenchymal, intravitreal, subretinal administration or for use in organ-targeted vascular delivery such as intraporal or intracoronary delivery or isolated limb perfusion. In certain embodiments the gene delivery vehicle of the first aspect of the invention, the combination according to the second aspect of the invention, or the pharmaceutical composition according to the invention is administered to the central nervous system, preferably by intracerebral injection, intraparenchymal injection, intrathecal injection, intra cisterna magna injection, intracerebroventricular injection or a combination thereof, more preferably by convection enhanced delivery.

In yet a further aspect, the invention provides for the gene delivery vehicle of the first aspect of the invention, the combination according to the second aspect of the invention or the pharmaceutical composition of the invention for use as a medicament. Preferably for use of a medicament for the treatment of a CNS disease such as Synucleinopathies, Huntington's Disease, tauopathies, Spinocerebellar ataxias, Amyotrophic Lateral Sclerosis and Frontotemporal dementia, Multiple System Atrophy, Lewy Body Dementia, Parkinson's Disease, PiD (Pick's disease), PSP (Progressive supranuclear palsy), CBD (corticobasal degeneration), AGD (Argyrophilic grain disease), AD (Alzheimer's disease), CTE (chronic traumatic encephalopathy), SCA1, SCA2 or SCA3 (Spinocerebellar ataxia 1, 2 or 3).

As explained herein, the gene delivery vehicle of the invention comprises at least one nucleic acid encoding an RNA that comprises a guide sequence that is substantially complementary to a target sequence in a gene associated with a disease and encodes an antibody of antigen binding fragment thereof that is targeting a protein associated with that same disease. In certain embodiments, the guide RNA is substantially complementary to a target gene as listed in Table 1 and the protein bound by the full-length mAb or an antigen binding fragment thereof is listed in Table 1.

TABLE 1
mRNA targets and protein targets for the gene delivery vehicles of the invention

| | | Umbrella | Specific |
|---|---|---|---|
| target gene | protein | indication | indication |
| SNCA | a-Syn | Synucleinopathies | Parkinson's Disease, |
| | | | Multiple System Atrophy |
| | | | Lewy Body Dementia |
| LRRK2 | a-Syn | Synucleinopathies | Parkinson's Disease |
| | | | Multiple System Atrophy |
| | | | Lewy Body Dementia |
| SNCA | LRRK2 | Synucleinopathies | Parkinson's Disease |
| | | | Multiple System Atrophy |
| | | | Lewy Body Dementia |
| TPPP/p25 | a-Syn | Synucleinopathies | Parkinson's Disease |
| | | | Multiple System Atrophy |
| | | | Lewy Body Dementia |
| SNCA | TPPP/p25 | Synucleinopathies | Parkinson's Disease |
| | | | Multiple System Atrophy |
| | | | Lewy Body Dementia |
| HTT | HTT | Huntington's Disease | |
| MSH3 | HTT | Huntington's Disease | |
| MAPT | TAU | Primary tauopathies | PiD (Pick's disease) |
| | | | PSP (Progressive supranuclear palsy) |
| | | | CBD (corticobasal degeneration) |
| | | | AGD (Argyrophilic grain disease) |
| APOE4 | TAU | Secondary tauopathies | AD (Alzheimer's disease) |
| MAPT | TAU | Secondary tauopathies | CTE (chronic |
| | | | traumaticencephalopathy) |
| ATXN1 | ATXN1 | Spinocerebellar ataxias | SCA1 |
| ATXN2 | ATXN2 | Spinocerebellar ataxias | SCA2 |
| ATXN3 | ATXN3 | Spinocerebellar ataxias | SCA3 |
| MSH3 | ATXN1 | Spinocerebellar ataxias | SCA1 |
| MSH3 | ATXN2 | Spinocerebellar ataxias | SCA2 |
| MSH3 | ATXN3 | Spinocerebellar ataxias | SCA3 |
| ATXN2 | TDP43 | Amyotrophic Lateral Sclerosis | |
| UBQLN2 | TDP43 | Amyotrophic Lateral Sclerosis | |
| C9ORF72 | TDP43 | Amyotrophic Lateral Sclerosis | |
| others | TDP43 | Amyotrophic Lateral Sclerosis | |
| FUS | FUS | Amyotrophic Lateral Sclerosis | |
| ATXN2 | TDP43 | Frontotemporal dementia | |
| UBQLN2 | TDP43 | Frontotemporal dementia | |
| C9ORF72 | TDP43 | Frontotemporal dementia | |
| others | TDP43 | Frontotemporal dementia | |
| FUS | FUS | Frontotemporal dementia | |

In preferred embodiments, the gene delivery vehicle of the first aspect of the invention, the combination according to the second aspect of the invention or the pharmaceutical composition of the invention are for use as a medicament for the treatment of synucleinopathies. Synucleinopathies are neurodegenerative disorders characterized by aggregation of alpha-Synuclein protein (α-Syn), encoded by the SNCA (Synuclein Alpha) gene. Accordingly, in preferred embodiments the guide RNA is substantially complementary to part of an alpha-synuclein (SNCA) gene and the antibody is an anti-alpha-synuclein antibody. In certain embodiments, the guide RNA is comprises or consists of SEQ ID NO: 9-15 and the antibody comprises:
a heavy chain variable domain of the antibody comprises an amino acid sequence with at least 95% sequence identity to SEQ ID NO's: 16 and a light chain variable domain of the antibody comprises an amino acid sequence with at least 95%, 96%, 97%, 98%, 99%, 100% sequence identity to at least one of SEQ ID NO's: 17; or
a heavy chain variable domain of the antibody comprises an amino acid sequence with at least 95% sequence identity to SEQ ID NO's: 18 and a light chain variable domain of the antibody comprises an amino acid sequence with at least 95%, 96%, 97%, 98%, 99%, 100% sequence identity to at least one of SEQ ID NO's: 19.

Preferably the antibody is the commercially available MED11341 or PRX002.

In a further aspect, the invention relates to a method for producing an expression cassette according to the invention, an AAV vector according to the invention, or a pharmaceutical composition according to the invention.

The methods for producing the nucleic acid of the invention comprise any methods for producing nucleic acids, including but not limited to de novo synthesis all of which would be apparent to the skilled person.

The method for producing an AAV vector preferably comprising the steps of: a) culturing a host cell as herein defined above under conditions such that the AAV vector is produced; and, b) optionally, one or more of recovery, purification and formulation of the AAV vector.

The host cell, preferably is a host cell that is suitable for the production of AAV vectors. Accordingly, the host cell is a host cell that is amenable to in vitro culture, preferably at large scale. Host cell that are suitable for the production of AAV vectors are well-known in the art and will typically be a mammalian or an insect cell line. Mammalian cell lines for producing AAV vectors are selected from among any mammalian species, including, without limitation, cells such as A549, WEHI, 3T3, 10T1/2, BHK, MDCK, COS 1, COS 7, BSC 1, BSC 40, BMT 10, VERO, W138, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumour cell. Mammalian cell lines for producing AAV vectors in particular include a broad range of HEK293 cell lines, of which the HEK293T cell line is preferred.

Insect cell lines for producing AAV vectors can be any cell line that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including rAAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SeIZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA).

Thus, in one embodiment, the expression cassette or construct is an insect cell-compatible vector or a mammalian cell-compatible vector. A “mammalian cell compatible vector” is understood to be a nucleic acid molecule capable of productive transformation or transfection of a mammalian cell or cell line. Mammalian cell-compatible vectors are well-known in the art. An “insect cell-compatible vector” is understood to be a nucleic acid molecule capable of productive transformation or transfection of an insect or insect cell. Exemplary insect cell compatible vectors include plasmids, linear nucleic acid molecules, and recombinant viruses, such as baculoviruses. Any vector can be employed as long as it is insect cell-compatible. The mammalian or insect cell-compatible vector may integrate into the cell's genome but the presence of the vector in the cell need not be permanent and transient episomal vectors are also included. The vectors can be introduced by any means known, for example by chemical treatment of the cells, electroporation, or infection.

The AAV in the supernatant can be recovered and/or purified using suitable techniques which are known to those of skill in the art. For example, monolith columns (e.g., in ion exchange, affinity or IMAC mode), chromatography (e.g., capture chromatography, fixed method chromatography, and expanded bed chromatography), centrifugation, filtration and precipitation, can be used for purification and concentration. These methods may be used alone or in combination. In one embodiment, capture chromatography methods, including column-based or membrane-based systems, are utilized in combination with filtration and precipitation. Suitable precipitation methods, e.g., utilizing polyethylene glycol (PEG) 8000 and NH₃SO₄, can be readily selected by one of skill in the art. Thereafter, the precipitate can be treated with benzonase and purified using suitable techniques. In addition, recovery may preferably comprises the step of affinity-purification of the (virions comprising the) recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74: 277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3 and AAV5 capsids.

In general, suitable methods for producing an AAV vector according to the invention in mammalian or insect host cells, and means, therefore (such as expression constructs for expression of AAV rep proteins), are described, for mammalian cells in: Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760), Xiao et al. (1998, J. Virol. 72, 2224-2232) and Judd et al. (Mol Ther Nucleic Acids. 2012; 1: e54), and for insect cells in: Urabe et al. (2002, Hum. Gene Ther. 13:1935-1943), WO2007/046703, WO2007/148971, WO2009/014445, WO2009/104964, WO2011/122950, WO2013/036118, WO2015/137802, WO2019/016349 and in co-pending applications EP21177449.2, PCT/EP2021/058794 and PCT/EP2021/058798, all of which are incorporated herein in their entirety.

In another aspect, the invention relates to a cell comprising the nucleic acid of the invention or the AAV of the invention, or a host cell.

In some embodiments, the cell of the invention is a prokaryote cell. In some specific embodiments, the cell of the invention is a bacterial cell. In some embodiments, the cell of the invention is a eukaryote cell. In some embodiments, the cell of the invention is a mammalian cell. In some embodiments, the cell of the invention is an insect cell.

The nucleic acid of the invention or the AAV vector of the invention may be delivered into the cell of the invention by any suitable methods, included but not limited to, transfection, transformation transduction, nucleofection, electroporation, microinjection. For example, the expression cassette or nucleic acid of the invention may be comprised in a plasmid contained in bacteria. The expression cassette or nucleic acid of the invention may also be comprised in a production cell that produces e.g. a viral vector. Said expression cassette may also be provided in a baculovirus vector.

In another aspect, the present invention relates to a method of treating or preventing a disorder, wherein the method comprises administering the nucleic acid of the invention or the AAV of the invention to a subject, thereby treating or preventing the disorder.

In another aspect, the invention relates to the nucleic acid of the invention or the AAV of the invention for use in the manufacture of a medicament for the treatment of a disorder as detailed herein.

The present invention has been described above with reference to a number of exemplary embodiments as shown in the drawings. Modifications and alternative implementations of some parts or elements are possible, and are included in the scope of protection as defined in the appended claims.

### BRIEF DESCRIPTION OF THE FIGURES

FIG. 1. miRNA plus antibody (mi-Ab) (A), miRNA (B) and antibody (vec-Ab) (C) construct design schemes

FIG. 2. Vectorized antibody levels in the culture medium and in cell pellets from transfected SH-SY5Y cells. Cells were transfected with all different versions of “vec-Ab” and “mi-Ab” constructs; mock transfection (Control) and non-aSyn-targeting antibodies (DOB1) were used as controls.

FIG. 3. A. Secreted Ab (total IgG levels) from transfected SH-SY5Y cells B. Average percentage of secretion from all types of constructs

FIG. 4. miSNCA5 expression levels from miSNCA and “mi-Ab” constructs in transfected SH-SY5Y cells.

FIG. 5. Dose-dependent expression of miSNCA5 in HEK293T cells after transfection with “vec-Ab” and “mi-Ab” constructs

FIG. 6. Dose-dependent expression of vectorized antibodies in HEK293T cells after transfection with “vec-Ab” and “mi-Ab” constructs A. MED11341 levels B. PRX002 levels

FIG. 7. Vectorized antibody levels A. in cell culture medium and B. in the cell extracts from SHSY-5Y cells transduced with AAV5-“vec-Ab” constructs

FIG. 8. Vector DNA (vDNA) levels in striatum and cortex from in vivo study 1

FIG. 9. Antibody mRNA levels, measured with universal assay, in the brain tissue samples from in vivo study 1. The mRNA levels in A. striatum and B. cortex

FIG. 10. Antibody levels, as measured by MSD, in the brain tissue samples from in vivo study 1. The antibody levels in A. striatum and B. cortex

FIG. 11. Antibody levels measured in serum samples 56 days after treatment

FIG. 12. Histological analysis of the brain sections from in vivo study 1. A. vector DNA (vDNA) ISH staining B. IHC staining for human antibody.

FIG. 13. Scheme depicting split of the brain and sectioning approach.

FIG. 14. Immunohistological analysis of spreading of a-Syn aggregates using pSer129 αSyn staining in striatum A) Area of immunoreactivity against phosphorylated alpha synuclein following IHC-staining. B) Optical density of phosphorylated alpha synuclein IHC staining. C) integrated optical density of phosphorylated alpha synuclein IHC staining. ** p≤0.01; *** p≤0.001 **** p≤0.0001.

### EXAMPLES

### Materials and Methods

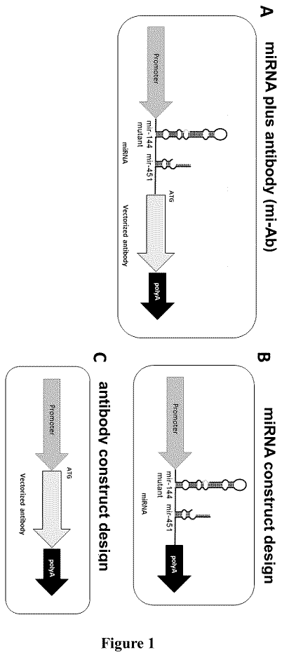

As defined herein “mi-Ab” constructs are constructs encoding a nucleic acid (such as an miRNA) and an antibody (full length or functional fragment thereof) (see FIG. 1A for a schematic representation).

miRNA Guide Strands Design

The miSNCAs (miRNA guide strands, FIG. 1B) were designed to target common RNA sequences (SEQ ID Nos 20-27) of the most common SNCA mRNA variants (SEQ ID NOs 28-31). A number of different guide strands having 22 nucleotides were generated (SEQ ID Nos 9-15) and incorporated into miR451 hairpin/scaffold (SEQ ID Nos 32) where “NNN” designates the guide strands and “YYY” designates the corresponding complementary sequences to the guide strands (SEQ ID NOs 9-15).

The miSNCA guides were selected based on the following criteria: the miRNA guide sequence should not include a stretch of >4 G, >4 C, >5 A and >5 T nt, a GC content between 30% and 70%, <4000 predicted off-target genes of the miRNA seed sequence for SNCA targeting guides and <5000 predicted off-target genes of the miRNA seed sequence for SNCA targeting guides by using siSPOTR analysis (https://sispotr.icts.uiowa.edu./sispotr/tools/lookup/evaluate.html) and pre-miRNA sequence folding energy between −44 and −55 kcal/mole. To generate a negative control, a non-targeting guide were selected for in vitro testing (miCTR1; miSCR(ANG1) (SEQ ID NO. 33).

The selected miSNCA guides meet the following criteria: conservation with monkey SNCA gene sequence (Macaca mulatta, NCBI accession number NC_041768.1), the miRNA guide sequence should not include a stretch of >4 G or >4 C nt, a GC content between 20% and 70%, a GC seed content between 40% and 70%, pre-miRNA sequence folding energy between −45 and −55 kcal/mole and no matching with endogenous miRNA seeds.

Guide sequences were incorporated into human pri-miRNA miR-451 scaffold sequences and the mFold program (http://unafold.rna.albany.edu/?q=mfold) was used with standard settings to determine whether the candidates are folded into the secondary structures.

The mi-part of the mi-Ab construct consists of the miR-144 hairpin/scaffold (SEQ ID NO 35) combined with one or more mir-451 downstream hairpin/scaffold (SEQ ID NOs 36-42), this way creating a mi-cassette for each selected miRNA (mi-cassette; SEQ ID NO 44): miSNCA2, miSNCA5, miSNCA7, miSNCA12, miSNCA13, miSNCA15, and miSNCA16 (SEQ ID NOs 45-52). Placement of the miR-144 hairpin is always at the 5′ compared to the miR451 hairpin sequence.

### Vectorized Antibody Design

For the vectorized antibody, “vec-Ab” (see FIG. 1C), we have chosen 2 antibodies from antibodies with sequences in the public domain. The heavy chain protein subunit (SEQ ID NOs 16 or 18) encoding DNA sequence (SEQ ID NOs 53 or 54) and light chain protein subunit (SEQ ID NO 17 or 19) encoding DNA sequence (SEQ ID NOs 55 or 56) were connected with a Furin cleavage site (F2A; SEQ ID NO 57).

As a negative control, DOB1 antibody which is directed towards Streptococcus pneumoniae 6B capsular polysaccharide was used. The heavy chain protein subunit (SEQ ID NOs 59) encoding DNA sequence (SEQ ID NO: 62) and the light chain protein subunit (SEQ ID NO 60) of DOB1 encoding DNA sequence (SEQ ID NO: 61) were connected with a F2A DNA sequence (SEQ ID NO 58) in between to create the “vec-Ab” version used in these examples.

The antibody design contained either a DNA sequence (SEQ ID NO 63) that encodes Ig leader peptide sequence (Ig-L; SEQ ID NO 64) or a DNA sequence (SEQ ID NO 65) that encodes human growth hormone leader (hGH-L; SEQ ID NO 66) as a signal peptide for efficient secretion.

For PRX002, MED11341 and DOB1; two different versions with these two signal peptide sequences were created (SEQ ID NOs 67-72).

Additionally, shorter scFv-Fc versions of full length antibodies were designed for MED11341, PRX002 and DOB1 (SEQ ID NOs 73-75).

“mi-Ab” Design

For combining miSNCAs and vec-Ab in one construct, the following strategy was used: the Alt promoter sequence (SEQ ID NO 76) was combined with an intron sequence (SEQ ID NO 77), and the mi-cassette miSNCA5 (SEQ ID NO 50) was inserted into this intron (“NNN” in the intron sequence refers to the miSNCAs cassette of miSNCA5. This intron was followed by one of the vec-Ab sequences of full length MED11341 or PRX002 that were generated. As a negative control construct, the miCTR1 cassette (SEQ ID NO 78) was inserted into the intron sequence and the vec-Ab construct of DOB1 antibody was inserted after the intron. In all cases, the SV40 polyA sequence (SEQ ID NO 8) was inserted at the end of the cassette to increase the stability of the mRNA from the mi-Ab constructs generated. In this way 6 different mi-Ab expression cassettes were generated (SEQ ID NOs 79-84).

### AAV Production

### DNA Constructs for Baculovirus Seed Generation

The expression cassettes carrying the different vec-Ab constructs were subcloned into ITRs containing plasmids generating pVD1562 (SEQ ID NO. 85), pVD1563 (SEQ ID NO. 86), pVD1564 (SEQ ID NO. 87), pVD1565 (SEQ ID NO. 88), pVD1566 (SEQ ID NO. 89), pVD1567 (SEQ ID NO. 90) (Table 2).

TABLE 2
List of the ITR containing plasmids generated.

| ID of ITR containing plasmids generated | The transgene |
|---|---|
| pVD1562 | Full length MEDI1341 with Ig Leader signal peptide |
| pVD1563 | Full length PRX002 Ig Leader signal peptide |
| pVD1564 | Full length MEDI1341 human growth hormone signal peptide |
| pVD1565 | Full length PRX002 human growth hormone signal peptide |
| pVD1566 | Full length DOB1 Ig Leader signal peptide |
| pVD1567 | Full length DOB1 human growth hormone signal peptide |

All these pVD plasmids carry Alt promoter, and an intron necessary for the promoter activity, followed by the vec-Ab constructs coding for full length antibody and the SV40 polyA sequence.

### AAV5 Vectors (Baculovirus Material)

RecombinantAAV5 harbouring the expression cassettes are produced by infecting SF+ insect cells (Protein Sciences Corporation, Meriden, Connecticut, USA) with two Baculoviruses, encoding Rep, Cap and Transgene. Following standard protein purification procedures on a fast protein liquid chromatography system (AKTA Explorer, GE 30 Healthcare) using AVB sepharose (GE Healthcare) the titer of the purified AAV was determined using QPCR.

### AAV5 Vectors (HEK Material)

AAV production by co-transfection of HEK 293T cells with a transfer vector and packaging plasmid(s) for the AAV5 serotype. Purification of the AAV was done via affinity chromatography & iodixanol gradient ultracentrifugation. After formulation and concentration, the titer of the purified AAV was determined using QPCR.

### Molecular, Biochemical and Histological Assays

DNA/RNA Isolation from In Vitro and In Vivo Samples

For the DNA and RNA isolation, AllPrep Mini kit from Qiagen (Catalog no. 80204) was used in all experiments described. Following manufacturer's protocol, RLT Plus lysis buffer was applied to the snap frozen cell pellets from in vitro cultures or pulverized animal tissue from in vivo samples. The cell/tissue suspension was then applied to Lysing Matrix D tubes with Matrix D beads (MP Biomedicals™ 116913100) and homogenized using TissueLyser system (Qiagen). The DNA and RNA were then isolated from this extract by applying it to first the DNA binding column from All Prep DNA/RNA mini kit. The filter through from DNA column was then added to the RNA binding column. After centrifugation and washing steps, DNA and RNA were eluted from the corresponding columns. This way DNA and RNA were isolated from the same sample. DNA and RNA quantity and integrity were determined by Nanodrop and Bioanalyzer.

Protein Extraction from In Vitro and In Vivo Samples

For protein isolations from in vitro samples, RIPA buffer (Sigma) containing PhosSTOP phosphatase inhibitor (Roche) and EDTA-free protease inhibitor (Roche) was used. For extraction of the protein the buffer was added into the cell pellet and the cells were agitated at 4 C, 400 rpm for 30 minutes. The cell extract was then centrifuged at top speed. The clarified supernatant was used for total protein measurement (BCA assay) and antibody detection using sandwich assay or the total IgG MSD assays that were developed in-house.

For protein isolations from in vivo samples, RIPA buffer (Sigma) containing PhosSTOP phosphatase inhibitor (Roche) and EDTA-free protease inhibitor (Roche) was applied to the pulverized animal tissue. The tissue suspension was then applied to Lysing Matrix D tubes (MP Biomedicals™ 116913100) and homogenized using TissueLyser system (Qiagen). After centrifugation at top speed the extract was used for total protein measurement and antibody detection using MSD assays.

Vector DNA Quantification from In Vitro and In Vivo Samples

DNA extraction from cell pellets or from pulverized brain tissue was performed using AllPrep DNA/RNA Mini Kit (Qiagen) and following the manufacturer's instructions. Vector genome copies were quantified by using TaqMan qPCR assay (Thermo Fisher Scientific) with primers/probe against the SV40 polyA region of the vector or primers/probe CMV enhancer region (Table 3). The quantification (GC/ug DNA) was done using linearized pVD plasmid to generate a standard curve with varying amounts of this linearized plasmid. The standard curve created this way was used to calculate the vector DNA copy number from the DNA isolated from cells transduced/transfected with the “vec-Ab”/miSNCAs/“mi-Ab” containing constructs and from in vivo tissue samples.

TABLE 3
Primer and probe sequences for the vDNA Taqman assays

| Assay | | | |
|---|---|---|---|
| for | Forward primer | Reverse primer | Probe sequence |
| SV40 | GGGAGGTGTGGGAGGTTT | AATGATTAACCCGCCATG | ACTTATCTACAGATCTGC |
| polyA | (SEQ ID NO. 91) | CT (SEQ ID NO. 92) | GGCCGCT |
| | | | (SEQ ID NO. 93) |
| CMV | AGTAACGCCAATAGGGACT | GGCGTACTTGGCATATGA | TTACGGTAAACTGCCCAC |
| enhancer | TTC | TACA | TTGGCA |
| | (SEQ ID NO. 94) | (SEQ ID NO. 95) | (SEQ ID NO. 96) |

RT-QPCR Assays for mRNA Quantification from In Vitro and In Vivo Samples

The cDNA syntheses were performed using the Maxima First Strand cDNA Synthesis Kit (Thermo Fisher Scientific) for RT-qPCR.

For the quantification of SNCA mRNA levels and vectorized antibody mRNA levels, RT-qPCR Taqman assays were used using a set of primers and probes designed against SNCA, MEDI1341 or PRX002 (Table 3). For MEDI1341 and PRX002 mRNA detection, sequence specific Taqman assays as well as a universal assay for detection of any vectorized human antibody were designed (Table 4).

TABLE 4
Primer sequences for RT-qPCR of HEK cells.

| Primer for | Forward primer | Reverse primer | Probe sequence |
|---|---|---|---|
| SNCA1 | AAATGTTGGAGGAGCAG | TCCAGAATTCCTTCCTGT | AGCAGGGAGCATTGCAG |
| | TGG | GG | CAGC |
| | (SEQ ID NO. 97) | (SEQ ID NO. 98) | (SEQ ID NO. 99) |
| Ab | CCGGAGAACAACTACAA | CACGGAGCATGAGAAGA | ACTCACCGTGGACAAGA |
| universal | GACCA | CGTT | GCAGGTGGCA |
| assay | (SEQ ID NO. 100) | (SEQ ID NO. 101) | (SEQ ID NO. 102) |
| MEDI1341 | CCTCTGCCTAAGTACCG | ACCATGCAGTAGTAGTC | ACAAGAGCGACGCCGAC |
| assay | GAT | GGC | AAGCACCA |
| | (SEQ ID NO. 103) | (SEQ ID NO. 104) | (SEQ ID NO. 105) |
| PRX002 | GATGACACAGAGCCCTA | ATGGTCAGGGTGAAGTC | AGCAGATTTTCTGGCAGC |
| assay | GCA | GGT | GGCTCTGGC |
| | (SEQ ID NO. 106) | (SEQ ID NO. 107) | (SEQ ID NO. 108) |

The RT-qPCR results were displayed as relative expression using the deltaCycle threshold (deltaCt) of the measured mRNA against the mean expression of 3 housekeeping genes; (Table 5).

TABLE 5
List of commercially available housekeeping
genes used for in vitro (human) and in vivo
(mouse) mRNA assays (from Applied Biosystems)

| Housekeeping | Assay | Used for |
|---|---|---|
| gene | ID | samples |
| Human GAPDH | ID: Hs02758991_g1 | Human (in vitro cell models) |
| Human GusB | ID: Hs00939627_m1 | Human (in vitro cell models) |
| Human ACTB | ID: Hs01060665_g1 | Human (in vitro cell models) |
| mATP5B | Mm01160389_g1 | Mouse (for in vivo studies) |
| mACTB | Mm01205647_g1 | Mouse (for in vivo studies) |
| mPGK1 | Mm00435617_m1 | Mouse (for in vivo studies) |

RT-QPCR Assay for miRNA Quantification from In Vitro and In Vivo Samples

To quantify miSNCA expression from miSNCA-constructs or “mi-Ab” constructs, a specific Taqman stem-loop-RT-qPCR miRNA assay (Thermo Fisher) was designed, specific for 23 nts miSNCA5. The assay ID for this Taqman assay (Thermo Fisher) is CTNKRV7. The expression levels were expressed as miRNA molecules/ug total RNA.

Electrochemiluminescence ELISA (MSD) for Quatification of Antibodies from In Vitro and In Vivo Samples

For measuring the amount of antibody in in vitro samples (cell medium and cell extracts) and in vivo samples (tissue extracts), two different MSD (Meso Scale Discovery) assays were developed. The first assay is a sandwich assay in which a sandwich between coating antibody, alpha synuclein protein and MEDI or PRX needs to be formed. The coating antibody is an N-terminal anti-human aSyn binding antibody (Mouse-a-aSyn 80-96, IgG1, kappa). A recombinant alpha synuclein protein (StressMarq; SPR-321E) is used as the substrate that both the coating antibody and the MED11341 or PRX002 can bind. Finally the detection of the MED11341 or PRX002 is done by addition of Sulfo-tag Anti-HU/NHP IgG antibody (MSD; D20JL-6) and reading at MSD scanner.

The second MSD assay was developed for measuring the amount of antibody in a sample is named as “total antibody MSD assay” of the human antibody. For this assay coating antibody that detects human Fc fragment (Goat anti-Human IgG-Fc Fragment Antibody IgG Fraction; A80-104 from Fortis Life Science) was used. Next the MEDI1341 or PRX002 antibody was applied to the coated plates. For detection Sulfo-tag Anti-Hu/NHP Kappa Light Antibody (MSD; D20TF-6) for PRX002 and Sulfo-tag Anti-Hu/NHP Lambda Light Antibody for MEDI1341 (MSD; D20QG-6) were used. Finally the detection of the signal was done in the MSD scanner.

In order to quantify the amount of Ab in samples, purified MEDI1341 or PRX002 antibodies were used to create a standard line between 0.1-10000 ng antibody/ml. A sigmoidal curve was fitted into log/log scale (Signal as absorbance unit versus amount of antibody in ng/mL) and the amount of antibody in each well were calculated based on this standard curve.

For each sample, total protein measurements were done using a bicinchoninic acid assay (BCA Protein assay kit; Pierce™). The MSD results were normalized by adding equal total protein into each well for the assay and the concentration of antibody is expressed as “ng/mL per ug protein”.

### Histopathology of Brain Tissue

Hematoxylin and eosin (H&E) stainings from brain sections (obtained from perfused fixated brain tissue) were done at contract research organization (Charles River Edinburgh Ltd) and histopathological examinations were done by a board-certified veterinary pathology expert.

In Situ Hybridization (ISH) for vDNA and Immunohisto Chemistry (IHC) for Total Human Antibodies in Brain tissue Samples

To evaluate the biodistribution of vDNA within the brain after intrastriatal brain injection, ISH probes (Assay ID: 894071-S1) against ITR regions of the vector DNA (designed by Advanced Cell Diagnostics) were used.

To evaluate the biodistribution of expressed antibody in the brain, IHC was performed using Goat anti-Human IgG Fc Cross-Adsorbed Secondary Antibody, HRP (Thermo Fisher Scientific cat #A18823).

### In Vitro Studies

### In Vitro Study 1

To measure the dose dependent expression of miSNCA5 in miSNCA-constructs and “mi-Ab” candidates and also to measure the expression of vectorized antibodies in “vec-Ab” and “mi-Ab” candidates, HEK293T cells were transfected with plasmids carrying corresponding constructs (Table 6). Three different doses corresponding to 5 fmol, 15 fmol and 50 fmol of plasmid were used for these experiments. The cells were kept for 3 days after transfection and on the last day the cell culture medium and cells were collected separately. The cell culture medium is collected, centrifugated to remove the dead cells and supernatants were kept at −80 C until MSD antibody measurements. The cells were collected by Accutase, centrifuged to get rid of the Accutase and the cell pellets were kept at −80 C until DNA/RNA or protein isolation.

TABLE 6
Plasmids carrying miSNCA-constructs, “vec-Ab”
and “mi-Ab” expression cassettes and used
for in vitro transfection of HEK293T cells.

| Construct | Plasmid | | SEQ |
|---|---|---|---|
| version | name | | ID NO |
| miSNCA- | miSNCA_5_w144 | aSyn003 | 109 |
| constructs | | | |
| miSNCA- | miCTR1 | aSyn001 | 78 |
| constructs | | | |
| “vec-Ab” | MEDI1341-IgLeader | | 125 |
| “vec-Ab” | PRX002-IgLeader | | 126 |
| “vec-Ab” | DOB1-IgLeader | | 127 |
| “vec-Ab” | MEDI1341-hGH-SP | | 128 |
| “vec-Ab” | PRX002-hGH-SP | | 129 |
| “vec-Ab” | DOB1-hGH-SP | | 130 |
| “vec-Ab” | ScFv-Fc-MEDI1341 | | 73 |
| “vec-Ab” | ScFv-Fc-PRX002 | | 74 |
| “vec-Ab” | ScFv-Fc-DOB1 | | 75 |
| “mi-Ab” | miSNCA5 + | aSyn013 | 110 |
| | MEDI1341-IgLeader | | |
| “mi-Ab” | miSNCA5 + | aSyn014 | 111 |
| | PRX-IgLeader | | |
| “mi-Ab” | miSNCA5 + | aSyn015 | 112 |
| | MEDI1341-hGH-SP | | |
| “mi-Ab” | miSNCA5 + | aSyn016 | 113 |
| | PRX002-hGH-SP | | |
| “mi-Ab” | miCTR1 + | aSyn021 | 114 |
| | DOB1-IGleader | | |
| “mi-Ab” | miCTR1 + | aSyn022 | 115 |
| | DOB1-hGH-SP | | |

### In Vitro Study 2

SHSY-5Y cells were transduced withAAV5-“vec-Ab” candidates (HEK cell production) at Multiplicity of infection (MOI) of 1⁵ for testing the antibody expression in AAV format.

### In Vivo Studies

### In Vivo Study 1—Dose-Range Study Vec-Ab in Wild Type Mice

In this study, the dose-dependent expression and safety of expressing vectorized antibodies after direct brain injection was evaluated. The two AAV-vec-Ab candidates (vec-Ab-PRX002 and vec-Ab-MEDI1341) were evaluated in wild type mice. A total of 7 treatment groups with 7 to 10 animals per group (total N=77) were used. On Day 1, all animals received a 2×3 μl bilateral injection of AAV5-vec-Ab-PRX002 or AAV5-vec-Ab-MEDI1341 into the striatum at three different doses (high, medium and low). The two groups injected with only formulation buffer or an empty AAV5 vector at medium dose served as control groups. Groups are indicated in Table 7.

TABLE 7
Study groups for the dose-range vec-Ab study

| | | | | | Collection Protocol |
|---|---|---|---|---|---|
| Group | Number of | Test | Titer | Dose | (animals per type |
| number | Animals | Article | (gc/mL) | (gc/brain) | of analysis) |
| 1 | 7 | Formulation | NA | NA | 3 Molecular Analysis |
| | | Buffer | | | 4 IHC/FISH |
| 2 | 10 | Empty Vector, | 7.0 × 10¹² | 4.2 × 10¹⁰ | 6 Molecular Analysis |
| | | mid dose | | | 4 IHC/FISH |
| 3 | 10 | AAV5-vec-Ab- | 7.42 × 10¹³ | 4.45 × 10¹¹ | 6 Molecular Analysis |
| | | MEDI1341, | | | 4 IHC/FISH |
| | | high dose | | | |
| 4 | 10 | AAV5-vec-Ab- | 1.44 × 10¹³ | 8.64 × 10¹⁰ | 6 Molecular Analysis |
| | | MEDI1341, | | | 4 IHC/FISH |
| | | mid dose | | | |
| 5 | 10 | AAV5-vec-Ab- | 2.56 × 10¹² | 1.54 × 10¹⁰ | 6 Molecular Analysis |
| | | MEDI1341, | | | 4 IHC/FISH |
| | | low dose | | | |
| 6 | 10 | AAV5-vec-Ab- | 5.37 × 10¹³ | 3.22 × 10¹¹ | 6 Molecular Analysis |
| | | PRX002, | | | 4 IHC/FISH |
| | | high dose | | | |
| 7 | 10 | AAV5-vec-Ab- | 9.13 × 10¹² | 5.48 × 10¹⁰ | 6 Molecular Analysis |
| | | PRX002, | | | 4 IHC/FISH |
| | | mid dose | | | |
| 8 | 10 | AAV5-vec-Ab- | 1.49 × 10¹² | 8.94 × 10⁹ | 6 Molecular Analysis |
| | | PRX002 | | | 4 IHC/FISH |
| | | low dose | | | |

Seventy-seven (77) male C57Bl/6J mice (Stock 000664) were purchased by PsychoGenics from Jax laboratory (8-9 weeks of age). Mice were group housed (3-4/cage) at PsychoGenics after health status clearance. Mice were assigned unique identification numbers and housed in polycarbonate OptiMICE cages. Each animal received a unique identification number, examined, manipulated and weighed prior to study initiation to ensure adequate health and suitability.

During the course of the study 12/12 light/dark cycles was maintained. The room temperature was maintained between 20 and 2300 with a relative humidity maintained around 50%. Chow and water was provided ad libitum for the duration of the study.

Mice were anesthetized with isoflurane and stereotactically injected bilaterally with test articles into the right and left forebrain to the dorsal neostriatum [3 μL per hemisphere; 6 μL per brain](coordinates: AP +0.8 mm, ML+/−1.8 mm, DV −3.0 mm). Injections were performed with a 10 μl syringe (Hamilton, NV) connected to a 30 G metal cannula using PE50 plastic tubing (PlasticsOne). Injections occurred at a rate of 0.5 μl/min with the needle left in place for 5 minutes following administration at each target. Pre- and post-operative analgesics was administered in accordance with veterinarian recommendations.

Animals were body weighed once per week and was checked for survival twice per day for the duration of the study, including weekends and holidays.

Experimental animals were sub mandibular bled at four time-points as shown in Table 8. Briefly, whole blood samples were processed for plasma using K2-EDTA tubes, flash frozen and stored at −80° C. until processing. For each time point, a single aliquot of approximately 25 μl was collected.

TABLE 8
Blood sample collection times

| Study | | | | | | | | | | |
|---|---|---|---|---|---|---|---|---|---|---|
| Timelines | | | | | | | | | | |
| (days) | −14 | −7 | 0 | 7 | 14 | 21 | 28 | 35 | 42 | 57 |

| | | | | | | | |
|---|---|---|---|---|---|---|---|
| Animals | X | | | | | | |
| Arrived | | | | | | | |
| Bilateral | | | X | | | | |
| Striatal | | | | | | | |
| injections | | | | | | | |
| Bleeds (Sub | | X | | X | X | X | |
| mandibular) | | | | | | | |
| Terminal | | | | | | | X |
| Biological | | | | | | | |
| Sample | | | | | | | |
| Collection | | | | | | | |

On day 57, animals were anesthetized with isoflurane and CSF, plasma, liver and brain samples were harvested.

CSF was collected from all animals. Briefly, the fur and skin from the neck was removed with surgical scissors (the mouse was not be decapitated). The tissue surrounding the base of the skull and brain stem was carefully removed via blunt dissection with minimal bleeding. Once the meninges were exposed at the base of the skull, in the area of the foremen magnum, a 27 G butterfly needle was held perpendicular to the skull and inserted laterally into the cerebrospinal fluid (CSF) space. Samples were centrifuged for 10 minutes at 2000 g, at 4° C. before freezing. Samples were stored in 0.5 mL tubes at −80° C. until use.

After CSF collections, whole blood (approximately 400 μl) was collected via closed cardiac puncture using a 1 ml syringe with 25-gauge needle prior to transcardial perfusion. Whole blood was processed for plasma by using K2-EDTA tubes. A single 200 μl aliquot of plasma was collected from each animal. Plasma samples were frozen on dry ice and stored at −80° C.

Animals were then flush perfused with saline until blood was fully cleared and tissue was harvested as described below:

Samples for molecular analysis (3 animals for group 1 and 6 animals/group for group 2-8): After the completion of the perfusion protocol, the liver was harvested and snap frozen (two vials with 100 mg of liver tissue from the left lateral lobe and one vial with the rest of the liver). Next, the whole brain was removed, weighed and micro-dissected into the frontal cortex, caudal cortex, striatum, cerebellum and the rest of the brain. The structures from both hemispheres were pooled into one vial and snap frozen.

Fixed samples for IHC/FISH (4 animals/group): After the completion of the perfusion protocol, the liver was harvested and post-fixed in 4% paraformaldehyde solution in a 30 mL vial for a minimum of 48 hours. Next, the brains were immediately harvested and placed into a 30 mL vial with 4% paraformaldehyde, the tissue to fixative ratio was at least 1:20. After 24 hours, the brain was dissected into two. Both pieces were placed back into 4% formaldehyde for a minimum of 12 hours and maximum of 24 hours.

After 48 hours, both the liver and brain pieces were then transferred to 70% ethanol, stored at 4° C. until paraffin embedding.

### In Vivo Study 2—Proof of Mechanism Study in PFF Mouse Model

In this study, the efficacy of vectorized antibodies and AAV-miSNCA was evaluated in M83 transgenic mice (expressing human A53T SNCA) injected with a-Syn pre-formed fibril (PFF). AAV vectorized antibody (vec-Ab-PRX002) or AAV-miSNCA were injected unilaterally into the M83 transgenic mouse brain at the start of the study (prophylactically on Day 1). The disease-causing agent human a-Syn PEE was then injected into the mouse striatum unilaterally to start the seeding of the a-Syn aggregates on Day 30. This pathology spreads through the brain after initial seeding. This spreading was then measured by immunohistochemistry of phosphorylated alpha synuclein. A total of 6 treatment groups with 14 animals per group (total N=84) were used. On Day 1, all animals received a 1×3 μl unilateral injection of AAV5-vec-Ab-PRX002 (high dose) or AAV5-miSNCA15 (high dose) into the striatum. There were 4 groups as controls: Group 1 (WT control), Group 2 (transgenic control), Group 5 (transgenic mouse injected with PEE disease model) and Group 6 (disease model with vectorized control antibody). Groups are indicated in Table 9.

TABLE 9
Study groups for the proof of mechanism in PFF mouse model

| | | | Treatment | | Unilateral | |
|---|---|---|---|---|---|---|
| | | | (Unilateral | | Striatal | |
| Group | | | Striatal) | Titer | Inoculum | Number |
| number | Group name | Genotype | Day 1 | (gc/brain) | Day 14 | of mice |
| 1 | WT_control | WT* | Formulation buffer | NA | PBS | 14 |
| 2 | Tg_control | TgM83+/−** | Formulation buffer | NA | PBS | 14 |
| 3 | AAV5-vec-Ab- | TgM83+/− | vec-Ab-PRX002 | 1.20E+11 | hPFF | 14 |
| | PRX002 | | high dose | | [2.5 mg/brain] | |
| 4 | AAV5-vec-Ab- | TgM83+/− | Vec-Ab-control | 1.20E+11 | hPFF | 14 |
| | control | | high dose | | [2.5 mg/brain] | |
| 5 | Vehicle_control | TgM83+/−** | Formulation buffer | NA | hPFF *** | 14 |
| | | | | | [2.5 mg/brain] | |
| 6 | AAV5-miSNCA15 | TgM83+/− | AAV-miSNCA15 | 1.20E+11 | hPFF | 14 |
| | | | high dose | | [2.5 mg/brain] | |

*WT; wild type mouse
**transgenic mouse hemizygous for human A53T-SNCA
*** human a-Syn PFF

### AAV Administration

Mice were anesthetized with isoflurane and stereotactically injected unilaterally with test articles or formulation buffer control into the dorsal neostriatum (3 μl in the right hemisphere) in an identical manner as described below. To minimize any pain or distress from surgery, all mice received analgesia both pre- and post-operatively according to recommendations from the veterinary team.

### PFF Inoculation

30 days (+/−1-3 days) after the AAV administration, mice were anesthetized with isoflurane and stereotactically injected in the right hemisphere with recombinant human α-synuclein fibrils (2.5 mg; 2.5 mg/μl) [unilateral injection]. Wild type and transgenic control animals received PBS. A single needle insertion (coordinates: +0.4 mm relative to Bregma, 2.2 mm from midline) into the right forebrain was used to target the inoculum to the dorsal neostriatum (+2.6 mm beneath the dura). Injections were performed using a 10 μl syringe (Hamilton, NV) at a rate of 0.1 μl per minute (1 μl total) with the needle left in place for ≥5 min following administration at each target. To minimize any pain or distress from the surgery, all mice received analgesia both pre and post-operatively according to guidance from the veterinary staff. Animals were monitored regularly following recovery from surgery.

### Body Weight and Survival

Animals were checked for survival twice per day and body weighed once a week.

### Terminal Collections

All animals were tissue collected at the end of the study (60 days post hPFF inoculation). Brains were harvested according to the methods described below:

Briefly, mice were deeply anesthetized with isoflurane and flush perfused with cold saline to remove blood cells until the liver was clear. Next, the brain was removed and divided into the right and left hemisphere. Each hemisphere was either fixed or micro dissected and frozen as below:

### IHC-Fixed:

Whole brain from seven animals per group were collected and drop fixed in freshly prepared 4% phosphate buffered PFA overnight at 4° C., then transferred to PBS and stored at 40 C until shipment or immunohistochemical processing. Before embedding, tissues were cryo-protected in 15% sucrose/PBS until sunk.

### Molecular Fresh Frozen:

Whole brains from seven animals per group were harvested and divided into the right and left hemisphere. Each hemisphere was further micro-dissected into the frontal cortex; posterior cortex; striatum; thalamus+hypothalamus; cerebellum+brainstem and the remainder of the brain tissue was snap frozen (−80° C.) separately. From all animals, the same region of the liver across groups (200/300100-150 mg) was also harvested and snap frozen (−80° C.)

Histology for Brain Samples [n=7 Per Group]

### Embedding

Immersion-fixed and cryo-protected brain samples were embedded in cryo molds within tissue freezing medium (OCT) and frozen on liquid, dry-ice cooled isopentane. To this end, a rostral and a caudal block, the rostral including the striatum, the caudal the substantia nigra (SN) were embedded side by side into one mold. Tissue blocks were stored at −80° C. until sectioning.

### Sectioning

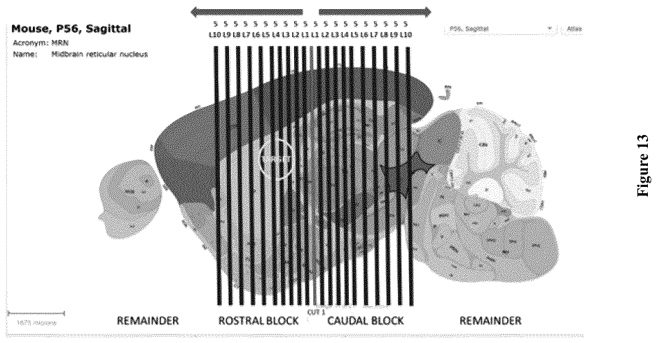

As shown in FIG. 13 brains were split using a matrix at approximately the frontal end of the hippocampus. The rostral and caudal blocks were embedded in parallel into one mold. After trimming, 5 serial sections were collected from 10 levels. Brain tissues (one hemisphere per animal) were cut coronally in a uniform systematic random protocol on a Leica CM3050 cryotome at 10 microns section thickness. In total 100 (50 sections each from the frontal and caudal brain parts) from 10 different coronal levels were collected per sample (5 consecutive sections per level). Collection of coronal sections started at the caudal end of the striatum and the frontal of the SN. During the first five levels (extension of the SN), 5 consecutive sections were kept, the next 5 sections were discarded in order to reach the next level. Then another five levels were collected throughout the striatum, always retaining 5 and discarding 35 sections. This way, collection extended through the entire substantia nigra and striatum. Sections were stored frozen until used.

### Analyses

### Labeling (Immunostaining)

Labeling was performed for all groups by immunostaining using specific markers. Systematic random sets of sections (8 sections from levels 1, 2, 3, 4, 5, 6, 8 and 10 of the brain) per mouse were labeled for: NeuN [1B7](Abcam, ab104224)+pSer129 αSyn [EP1536Y](Abcam, ab51253).

All labelled samples were counterstained with DAPI to visualize nuclei. Primary antibody binding was visualized using highly cross-absorbed fluorescently labeled secondary antibodies. Standard operational negative controls (OPNCs) included sections running through the whole protocol but without the incubation with the primary antibody. Entire brain sections were imaged on a Zeiss AxioScan.Z1 slide microscope scanner equipped with LED illumination, corresponding narrow band filter sets fitting to each dye and a sensitive Orca Flash 4.0 B&W camera at 10× magnification (mounted with 1× opto-coupler thus no demagnification).

### Quantifications

The following variables were quantified in the dorsal striatum and substantia nigra of both hemispheres.
a) Region size
b) Immunoreactivity (IR) area, object density (O.D.), mean signal, mean size and Integrated optical density (IOD) of all markers
c) De-biased neuron density

Regions of interest (ROIs) were manually delineated to determine region size, which served for normalization of IR surface area to the individual ROI. Cavalieri volume for determination of atrophy was estimated using the individual ROI sizes. Quantifications using the stored ROIs were done blind and fully automated using Image Pro Premier software (v 9.1.5 or higher).

### Statistical Analysis

Data were analyzed by analysis of variance (ANOVA) followed by post-hoc comparisons where appropriate. An effect was considered significant if p<0.05. Data were represented as the mean and standard error to the mean (s.e.m). Histological data were compared by One-way ANOVA followed by Fisher's LSD posttest.

### Molecular Analysis

The snap frozen tissue from the frontal cortex; posterior cortex; striatum; thalamus+hypothalamus; cerebellum+brainstem of each hemisphere of 7 animals from each group are separately processed for molecular analysis. The tissues are pulverized and aliquoted into batches for DNA/RNA isolation and protein isolation. The DNA and RNA is isolated using an AllPrep DNA RNA isolation kit from Qiagen according to manufacturer's instructions. Vector DNA levels are measured using specific TaqMan assays. SNCA mRNA levels are measured using a human SNCA specific TaqMan assay. Aggregated alpha synuclein are isolated from frontal cortex and striatum samples and the levels are measured using MSD (Meso Scale Discovery) and ELISA assays. Total alpha synuclein levels are measured in these protein isolates using U-PLEX Human α-Synuclein Kit from Meso Scale Discoveries. Vectorized antibody levels are measured in protein isolates from cortex, striatum, brainstem and cerebellum tissue isolates using MSD/ELISA assays optimized for detection of human antibody.

### In Vivo Study 3—Dose-Response Study in PFF Mouse Model

Several doses of the most potent AAV-miSNCA, AAV-vec-Ab and/or AAV-mi-Ab candidates are tested in the a-Syn PFF mouse model.

### Results

### In Vitro Studies (Transfection)

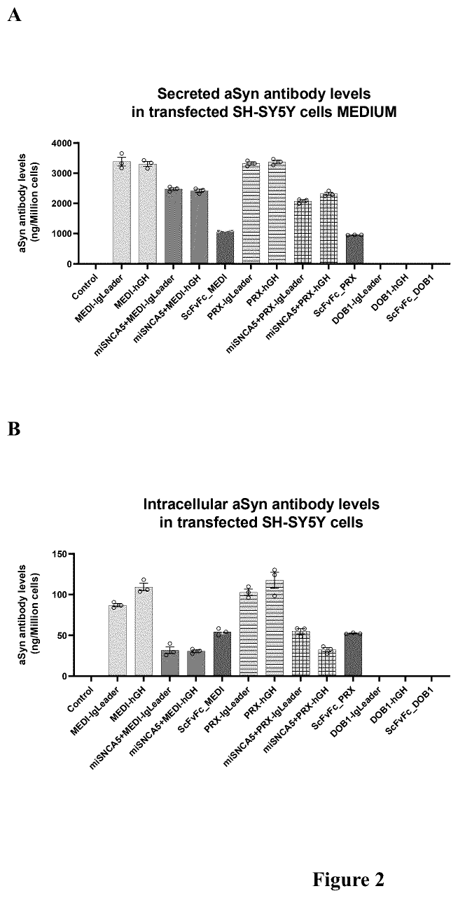

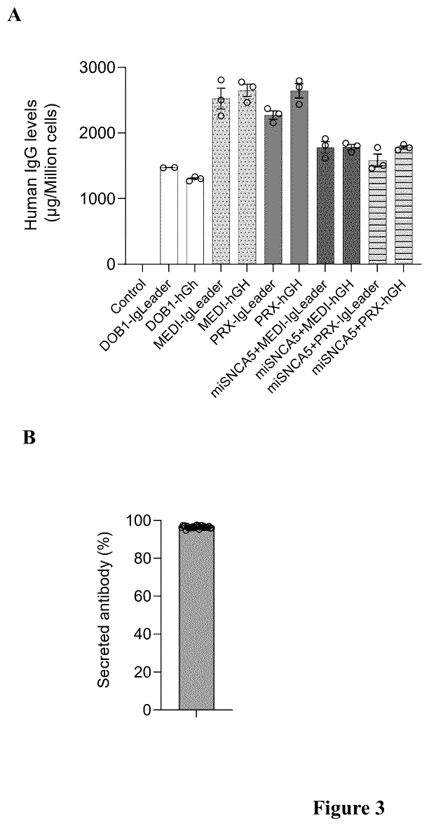

All versions of vec-Ab and mi-Ab encoded in plasmids, led to expression of antibodies after cell transfection. The antibodies could be detected both in the cell medium and in the cell pellets (FIGS. 2A and B). The full length and ScFv-Fc short versions of the antibodies were expressed and were functional as shown in the MSD sandwich assay. Most of the antibodies expressed were secreted efficiently in vitro. SH-SY5Y cells were transfected with vec-Ab and mi-Ab containing plasmids. All of the constructs expressed and secreted the antibodies (FIG. 3A). Antibody levels were measured in cells and culture medium with MSD and over 96% of the antibodies are secreted into the medium (FIG. 3B).

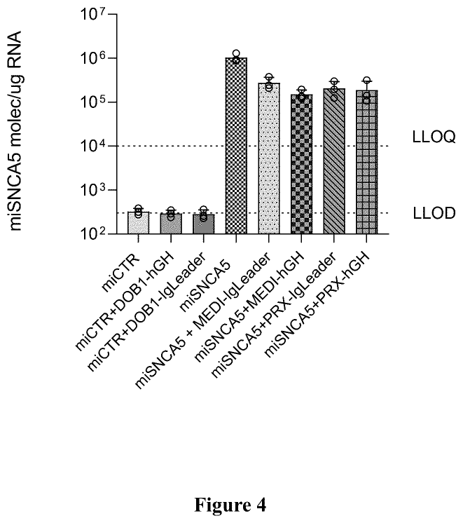

miSNCA5 was expressed from miSNCA and mi-Ab constructs in SHSY5Y cells after transfection (single dose tested) (FIG. 4).

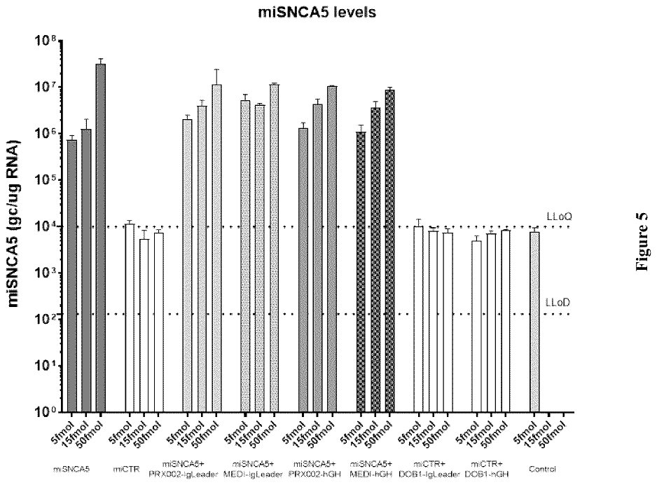

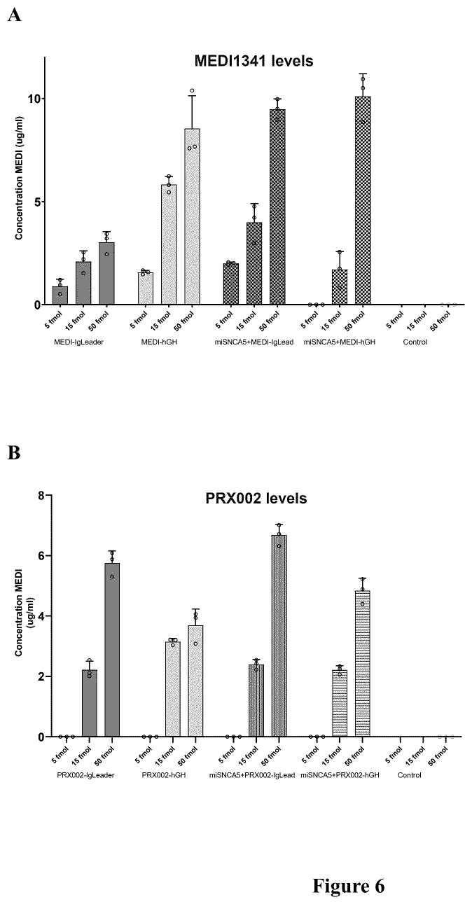

A dose-dependent expression was shown in HEK293T cells after transfection for both the miSNCA5 (FIG. 5) and vec-Ab constructs (FIG. 6). All of the vec-Ab and mi-Ab constructs tested led to antibody expression in the cells.

### In Vitro Studies (Transduction)

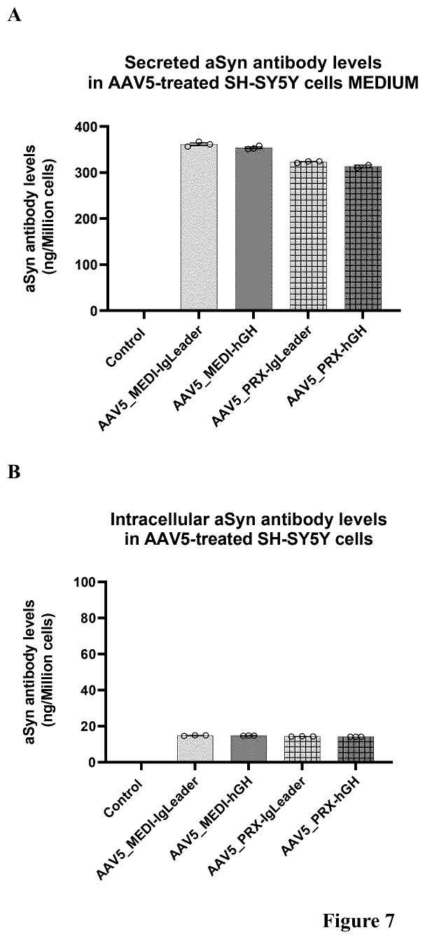

AAV5-vec-Ab candidates is shown expression of the corresponding vectorized antibodies The experiments show that the antibodies can be expressed from vec-Ab cassette also from AAVs. Vectorized antibodies were expressed and efficiently secreted in to the medium (FIG. 7A). The antibodies expressed were also detected in the cell extracts as shown in FIG. 7B.

### In Vivo Studies

### In Vivo Study 1

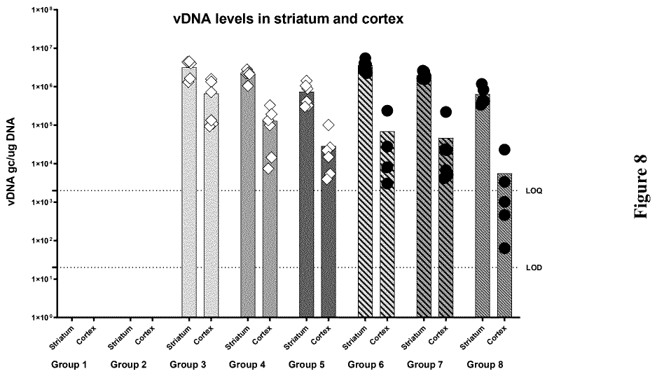

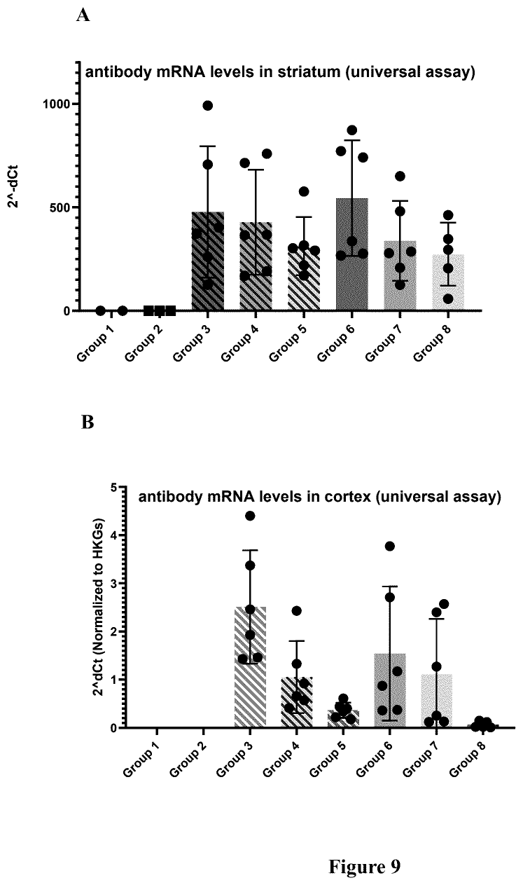

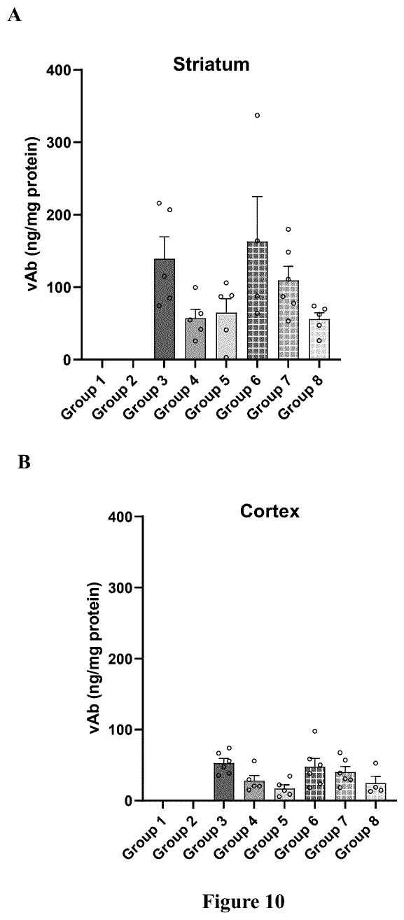

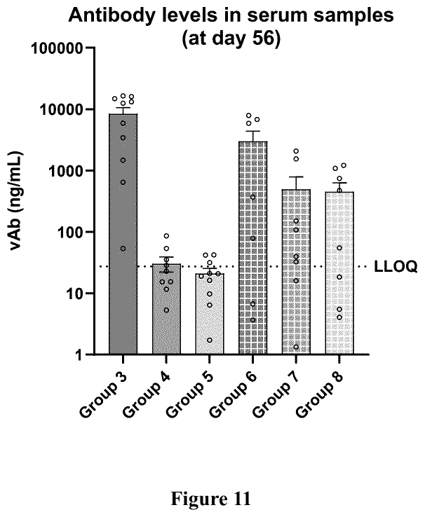

To address the adequacy of AAV vectors to deliver vec-Ab candidates to target brain regions, AAV5 was chosen as a vector to deliver vec-Ab via intrastriatal administration. Antibody expression levels and histopathology were assessed after AAV5-vec-Ab administration to the striatum in adult wt mice. Dose-dependent vDNA levels were observed both in striatal and cortical samples (FIG. 8). Accordingly, a dose-dependent expression of the corresponding antibody-coding mRNA was detected in both striatum and cortex (FIGS. 9A&B). a-Syn binding antibodies were detected both in striatal and cortical samples (FIGS. 10A&B). In the high-dose treatment groups (Group 3 and Group 6) the antibody could also be detected in the serum samples (FIG. 11). Baseline serum samples from these group of animals were all undetected.

FIG. 11 shows the vectorized antibody values from serum samples in different treatment groups. The data in graph is presented in log scale to better visualize the differences between measurements.

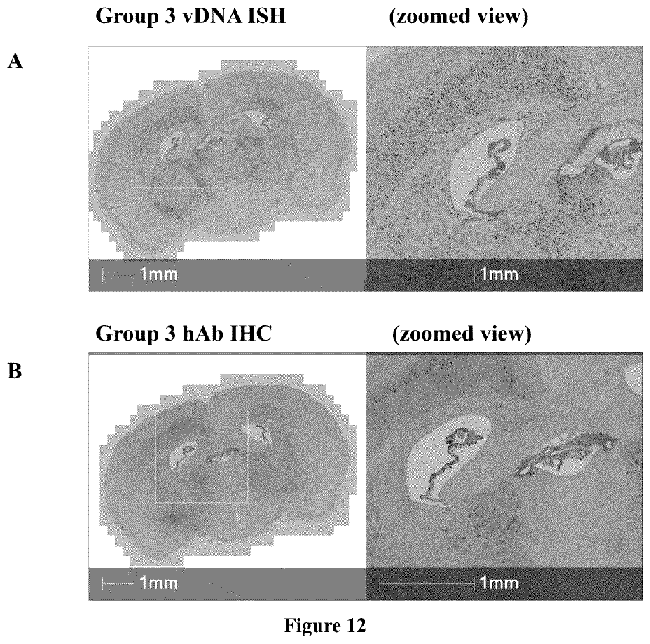

The vDNA ISH staining (FIG. 12A) and human antibody IHC stainings (FIG. 12B) showed a good biodistribution to the striatum and cortex.

The H&E stainings showed that the expression of the antibodies did not induce any safety concerns according to the pathology report.

The current in vivo study established safe and dose-dependent expression of vectorized a-Syn antibodies after direct brain injections in WT mouse. The antibodies are expressed and secreted efficiently and can be found in the CSF which might help increase delivery of the therapy to all brain regions besides the transduced regions.

### In Vivo Study 2

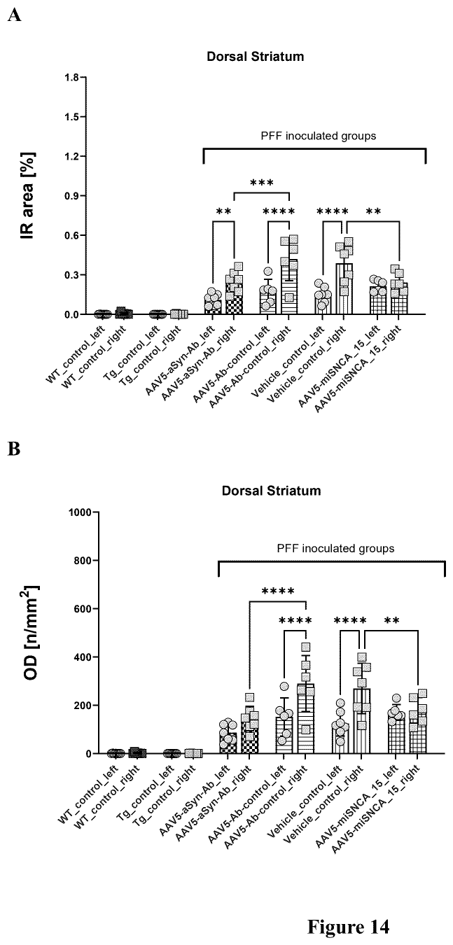

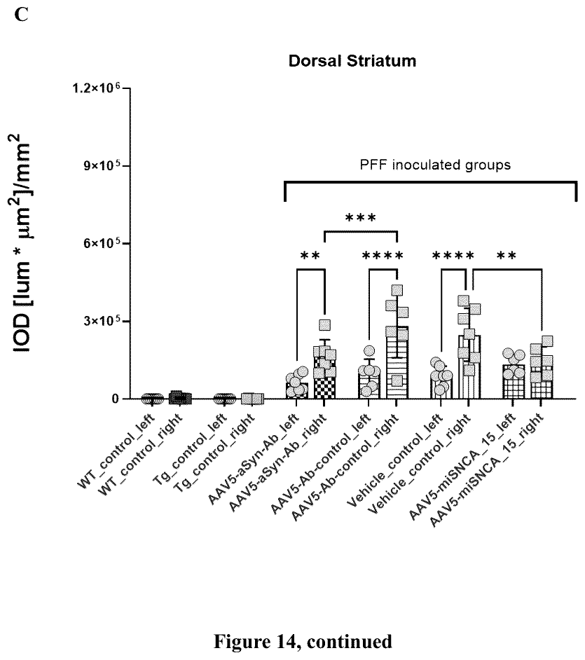

To assess the effects of miSNCA and vectorized antibody expression on the spreading of a-Syn aggregates, M83 mice injected with a-Syn PFF were employed. The decrease of spreading was measured as suppression of the spread from right to left hemisphere using phosphorylated alpha synuclein (pSer129 αSyn) IHC in striatum and cortex regions as a read out. For quantification of the spread in these regions, IR area, optical density (OD), mean signal, mean size and integrated optical density (IOD) of phosphorylated a-Syn were assessed in the striatum. All measurements of phosphorylated a-Syn in striatum (IR, O.D. and I.O.D.) showed a significant decrease in the spread of a-Syn aggregates in the aSyn vectorized antibody (AAV5-αSyn-Ab) group as compared to the vectorized antibody control (AAV5-Ab-control) group (FIGS. 14A, 14B and 14C). Similarly, AAV5-miSNCA15 led to reduced phosphorylated a-Syn when compared with the vehicle control group in striatum (FIGS. 14A, 14B and 14C). For each study group there are two bars showing left and right hemisphere for pSer129 αSyn staining. It can be appreciated that in the aSyn antibody condition IR area, O.D. and I.O.D. measured in the right brain are all lower compared to the control antibody condition. A similar result was observed for miSNCA and vehicle control. Any phosphorylated alpha synuclein detected in the left hemisphere has spread there from the right hemisphere. Of note, especially the aSyn antibody condition shows clear inhibition of the spread to the left hemisphere as compared to the control antibody condition. These results indicate that combining both an antibody and a microRNA in one construct enables both inhibition of newly forming fibrils using miSNCA, while at the same time degrading any pre-existing or newly arising fibrils.

1. A gene delivery vehicle comprising an expression cassette
comprising at least one nucleic acid comprising a sequence encoding a first RNA and a sequence encoding a second RNA, wherein the second RNA comprises a guide sequence and wherein the first and second RNA each comprise a hairpin,
and encoding a full-length mAb or an antigen binding fragment thereof,
wherein the guide sequence is substantially complementary to a target sequence in a gene associated with a disease and wherein the mAb or antigen binding fragment thereof is targeting a protein associated with that same disease.

2. The gene delivery vehicle according to claim 1, wherein the nucleic acid encodes at least one further RNA preferably comprising a further guide sequence.

3. A combination of two or more gene delivery vehicles comprising:
a first gene delivery vehicle comprising a first expression cassette comprising at least one nucleic acid comprising a sequence encoding a first RNA and a sequence encoding a second RNA, wherein the second RNA comprises a guide sequence and wherein the first and second RNA each comprise a hairpin; and
a second gene delivery vehicle comprising a second expression cassette, comprising a transgene encoding a full-length mAb or an antigen binding fragment thereof,
wherein the guide sequence is substantially complementary to a target sequence in a gene associated with a disease and wherein the mAb or antigen binding fragment thereof is targeting a protein associated with that same disease.

4. The combination of gene delivery vehicles according to claim 3, wherein the combination further comprises at least one further gene delivery vehicle comprising at least one further expression cassette comprising at least one further nucleic acid sequence encoding a further RNA, preferably comprising a further guide sequence.

5. The gene delivery vehicle according to claim 1 or the combination of gene delivery vehicles according to claim 3, wherein the guide sequence is substantially complementary to a target sequence in a gene that encodes the antigen bound by the full-length mAb or an antigen binding fragment thereof.

6. The gene delivery vehicle according to claim 1 or the combination according to claim 3, wherein, in a 5′ to 3′ direction, the sequence encoding the first RNA is followed by a spacer, which spacer is followed by the sequence encoding the second RNA, and wherein the spacer is at least 15 nucleotides.

7. The gene delivery vehicle according to claim 1 or the combination according to claim 3, wherein the hairpin in the second RNA comprises at least 40 nucleotides.

8. The gene delivery vehicle according to claim 1 or the combination according to claim 3, wherein the expression cassette is a DNA molecule.

9. The gene delivery vehicle according to claim 1, or the combination according to claim 3, wherein the expression cassette further comprises a promoter that is operably linked to the nucleic acid and optionally further comprises a poly-A signal.

10. The gene delivery vehicle according to claim 1 or the combination according to claim 3, wherein the expression cassette is flanked by at least one adeno-associated virus (AAV) Inverted Terminal Repeat (ITR).

11. The gene delivery vehicle according to claim 1 or the combination according to claim 3, wherein the gene delivery vehicle is an AAV vector.

12. The gene delivery vehicle or combination according to claim 11, comprising an AAV5 or AAV9 capsid protein.

13. The gene delivery vehicle according to claim 1 or the combination according to claim 3, wherein the first second and further RNA comprise a sequence selected from the group consisting of SEQ ID NO: 2, 3, 4 and 119; and variants thereof.

14. A pharmaceutical composition comprising the gene delivery vehicle according to claim 1 or the combination according to claim 3 and at least one pharmaceutically acceptable excipient.

15. (canceled)
